(19) United States
(12) Patent Application Publication
(10) Pub. No.: US 2024/0009151 A1
(43) Pub. Date: Jan. 11, 2024

(54) FORMULATIONS OF AZOBENZENE PHOTOREACTIVE COMPOUNDS

(71) Applicants: BAYON THERAPEUTICS, INC., Solana Beach, CA (US); The Regents of the University of California, Oakland, CA (US)

(72) Inventors: Richard H. KRAMER, Oakland, CA (US); Kevin CAO, Oakland, CA (US); Hovhannes GUKASYAN, Solana Beach, CA (US); Brian M. STREM, Solana Beach, CA (US)

(21) Appl. No.: 18/250,494

(22) PCT Filed: Oct. 22, 2021
(86) PCT No.: PCT/US2021/056323

§ 371 Date: Apr. 25, 2023

Related U.S. Application Data
(60) Provisional application No. 63/106,297, filed on Oct. 27, 2020.

(51) Int. Cl. A61K 31/167; A61K 47/40; A61P 27/02; A61K 9/00

(52) U.S. Cl. CPC A61K 31/167; A61K 47/40; A61P 27/02; A61K 9/0019

(57) ABSTRACT

Disclosed herein are pharmaceutical compositions comprising an azobenzene photoswitch compound and an alkylated cyclodextrin. Such compositions may be administered to the eye to treat a variety of retina disorders.

### BACKGROUND

### Field

The present disclosure relates to pharmaceutical formulations of azobenzene compounds for the treatment of retinal disorders.

### Description of the Related Art

Certain photochromic azobenzene compounds exhibit photoswitch behavior, whereby the compounds undergo photoisomerization, changing the length and geometry of the compounds. Such compounds can interact with proteins, and thereby alter protein function when the compounds undergo photoisomerization. These compounds have been shown to confer light-sensitivity to degenerated retinas by interaction with membrane proteins in retinal ganglion cells and upstream neurons. Therefore, such compounds can be used to improve vision in patients suffering from retinal disorders. However, many of these compounds are poorly soluble and do not disperse well within the eye after administration. Accordingly, there is a need for improved formulations of azobenzene compounds.

### SUMMARY

Some embodiments disclosed herein include a pharmaceutical composition, comprising:
an alkylated cyclodextrin; and
a compound of formula (I):

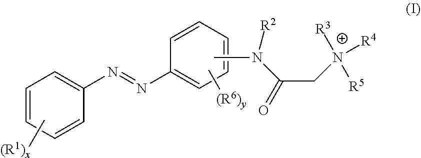

wherein each of R₁ are independently selected from C₁₋₁₀ alkyl, substituted C₁₋₁₀ alkyl, —NR¹⁰R¹¹, —NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl; substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, —C(O)OR¹⁰, and —S(O)₂R¹⁰;
x is an integer from 0 to 5;
y is an integer from 0 to 4;
R² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
R³, R⁴, and R⁵ are independently selected from hydrogen, C₂₋₈ alkyl, substituted C₂₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
each R⁶ is independently selected from hydrogen, C₁₋₁₀ alkyl, substituted C₁₋₁₀ alkyl, NR¹⁰R¹¹, —NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, —C(O)OR¹⁰, —S(O)R¹⁰, and —S(O)₂R¹⁰;
R¹⁰ and R¹¹ are independently selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
R¹² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl; and
R¹³ is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆-C₁₀ aryl, substituted C₆₋₂₀ aryl, C₄₋₁₀ cycloalkyl, substituted C₄₋₁₀ cycloalkyl, C₄₋₁₀ cycloalkenyl, substituted C₄₋₁₀ cycloalkenyl, —CH₂—N(CH₂CH₃)₃⁺, and —CH₂—SO₃⁺,
or a pharmaceutically acceptable salt thereof;
wherein the azo bond in the compound of formula (I) may be either cis or trans; and
wherein the molar ratio of cyclodextrin to compound of formula (I) is from 1:1 to 500:1.

Some embodiments include a method of treating a retinal disorder, comprising injecting the composition into the vitreous of a subject having the retinal disorder.

### BRIEF DESCRIPTION OF THE DRAWINGS

FIG. 1A shows spike rasters and firing frequency plots of retinal ganglion cells.

FIG. 1B shows histograms of light response indices.

FIG. 1C shows normalized histograms comparing relative change in light responsive retinal ganglion cells.

FIG. 1D is a graph showing correlation of light response indices with amount of SBE-CD.

FIG. 2A depicts a schematic of a 2-photon microscope.

FIG. 2B shows micrographs of light responsive cells in retinas.

FIG. 2C shows overlays of correlation micrographs.

FIG. 2D is a plot of the fraction of light responsive cells from retinas.

FIG. 2E shows activity rasters.

FIG. 3A shows spike rasters and frequency plots from retinas.

FIG. 3B shows spike rasters and frequency plots from retinas after isolation of retinal ganglion cells.

FIG. 3C is a plot showing the time course of light responsive indices.

FIG. 3D are graphs comparing photosensitization time courses.

FIG. 3E is a scatter plot correlating light responses with compound abundance.

FIG. 4A shows spatial light responsive index plots and pictures of retinas.

FIG. 4B shows correlation micrographs of retinas.

FIG. 4C is a plot of fraction of light responsive cells as a function of distance.

FIG. 4D depicts bar plots showing consistency of light response.

FIG. 5A is a plot of fraction of light responsive cells as a function of distance.

FIG. 5B depicts bar plots showing consistency of light response.

### DETAILED DESCRIPTION

Various embodiments described herein include pharmaceutical compositions that include a cyclodextrin and an azobenzene photoreactive compound. In some embodiments, the cyclodextrin is an alkylated cyclodextrin. These compositions can be administered to a subject, such as by intravitreal injection, to treat various disorders of the eye, including retinal disorders such as retinitis pigmenosa or age-related macular degeneration. In various embodiments, the composition can provide fully soluble aqueous solutions that achieve uniform tissue distribution of the azobenzene compound in the eye and prolongation of the photosensitizing effect.

### Azobenzene Compounds

Azobenzene photoreactive compounds suitable for use as described herein can include those described in International Application Publication No. WO/2010/051343, which is incorporated herein by reference in its entirety. In some embodiments, the azobenzene compounds have the structure of Formula (I):

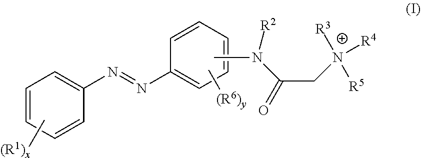

wherein each of R₁ are independently selected from C₁₋₁₀ alkyl, substituted C₁₋₁₀ alkyl, —NR¹⁰R¹¹, —NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl; substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, —C(O)OR¹⁰, and —S(O)₂R¹⁰;
x is an integer from 0 to 5;
y is an integer from 0 to 4;
R² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
R³, R⁴, and R⁵ are independently selected from hydrogen, C₂₋₈ alkyl, substituted C₂₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
each R⁶ is independently selected from hydrogen, C₁₋₁₀ alkyl, substituted C₁₋₁₀ alkyl, NR¹⁰R¹¹, —NR²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, —C(O)OR¹⁰, —S(O)R¹⁰, and —S(O)₂R¹⁰;
R¹⁰ and R¹¹ are independently selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
R¹² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl; and
R¹³ is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆-C₁₀ aryl, substituted C₆₋₂₀ aryl, C₄₋₁₀ cycloalkyl, substituted C₄₋₁₀ cycloalkyl, C₄₋₁₀ cycloalkenyl, substituted C₄₋₁₀ cycloalkenyl, —CH₂—N(CH₂CH₃)₃⁺, and —CH₂—SO₃⁻,
or a pharmaceutically acceptable salt thereof.

In some embodiments, each R₁ is independently selected from halo, C₁₋₁₀ alkyl, —NR¹⁰R¹¹, and —NR¹²C(O)R¹³.

In any of the foregoing embodiments, R² can be hydrogen or ethyl.

In any of the foregoing embodiments, x can be 0 or 1.

In any of the foregoing embodiments, y can be 0 or 1.

In any of the foregoing embodiments, R³, R⁴, and R⁵ can each be ethyl.

In any of the foregoing embodiments, R⁶ can be halo or C₁₋₁₀ alkyl.

In any of the foregoing embodiments, R¹⁰ and R¹¹ can independently selected from hydrogen, C₁₋₁₀ alkyl, and substituted C₁₋₁₀ alkyl;

In any of the foregoing embodiments, R¹² can be hydrogen.

In any of the foregoing embodiments, R¹³ can be selected from C₁₋₁₀ alkyl, substituted C₁₋₁₀ alkyl, C₂₋₈ alkenyl, and C₆₋₁₀ aryl.

Although Formula (I) depicts an azo bond in the trans configuration, it is to be understood that the formula encompasses both the cis and trans configuration, unless otherwise indicated herein.

Some specific embodiments of Formula (I) include the following structures:

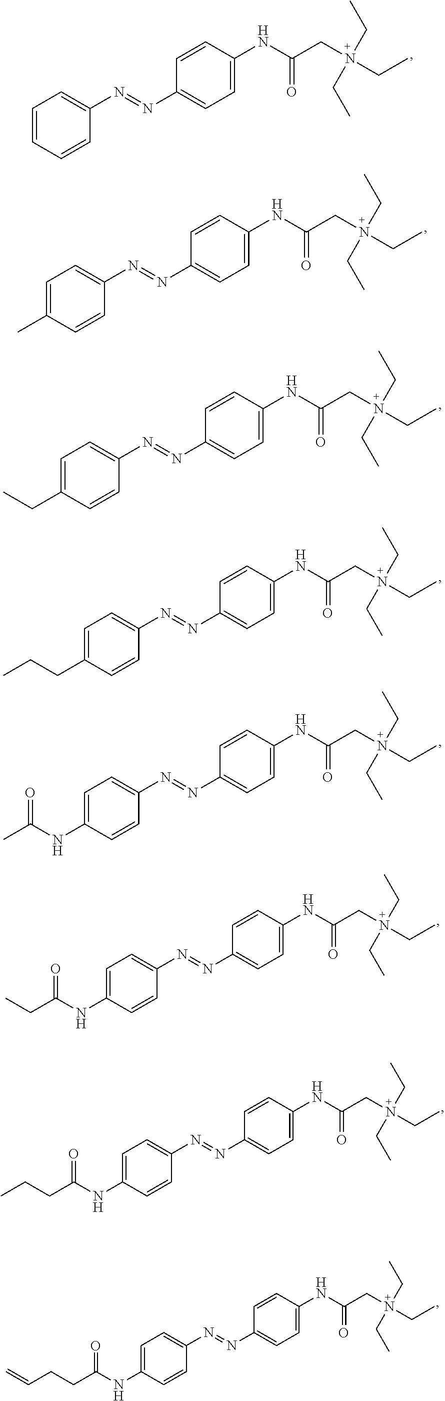

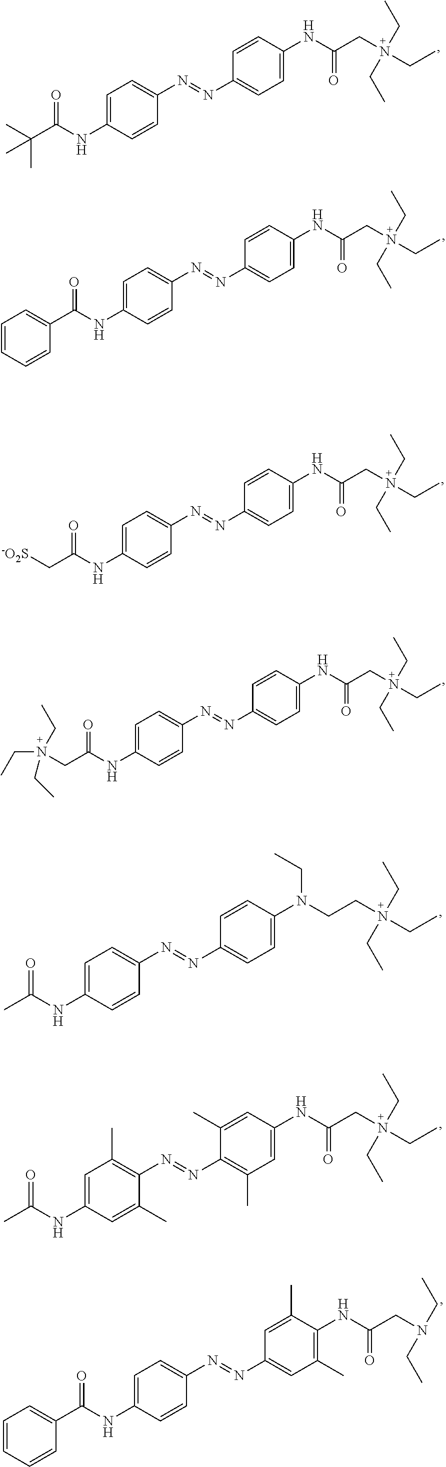

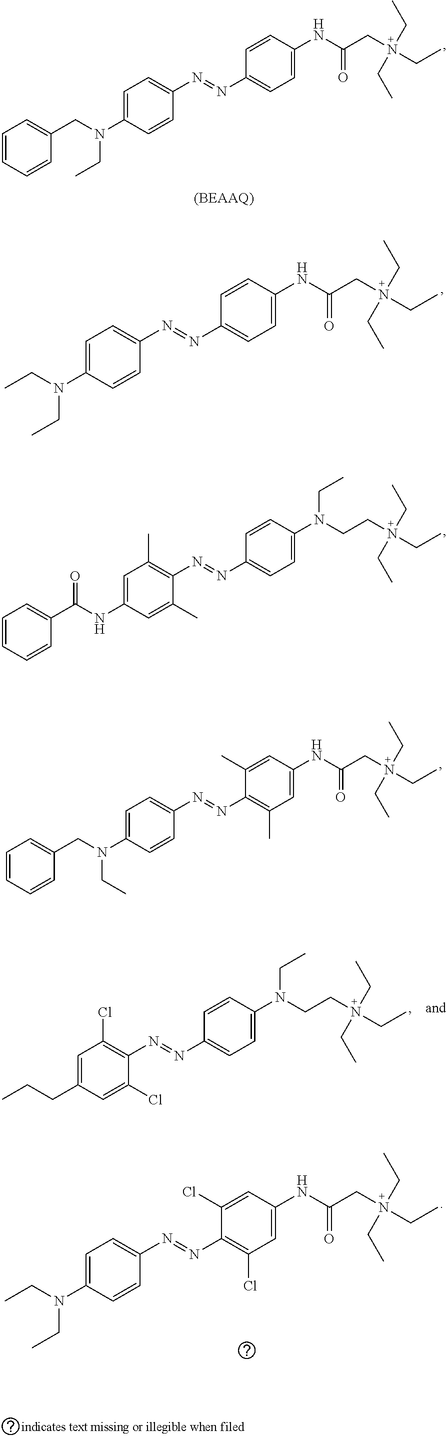

or pharmaceutically acceptable salts thereof, wherein the azo bond in the compounds of formula (I) may be either cis or trans unless otherwise indicated herein.

In some embodiments, the compound of Formula (I) has the structure of Compound 1:

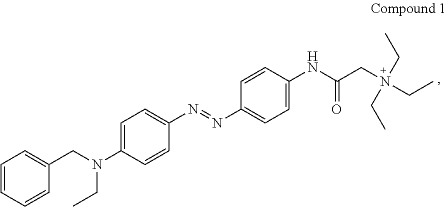

or pharmaceutically acceptable salts thereof, wherein the azo bond in the structure may be either cis or trans.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to a straight- or branched-chain alkyl group having from 1 to 12 carbon atoms in the chain, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples.

As used herein, “alkoxy” refers to the formula —OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “ C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “ C₃₋₆ carbocyclyl” or similar designations. Examples of carbocyclyl rings include, but are not limited to, cyclopropyl, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to —C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O)OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O)OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(═O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamide” group refers to a “—N(R_{A})SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A})C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more subsitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, quaternary ammonium, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH_{2—, —}CH₂CH₂—, —CH₂CH(CH₃)CH₂—, and the like. Other radical naming conventions clearly indicate that the radical is a di-radical such as “alkylene” or “alkenylene.”

### Alkylated Cyclodextrin

As used herein, an “alkylated cyclodextrin” is a cyclodextrin wherein one or more hydrogen atoms on the hydroxy substituents on the cyclodextrin are replaced with an alkyl group, which may be optionally substituted with other substituents. In one embodiment, alkylated cyclodextrins for use as described herein have the structure of Formula (II):

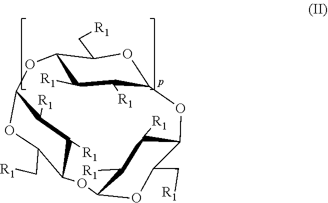

or pharmaceutically acceptable salts thereof, wherein p is 4, 5, or 6, and R₁ is independently selected at each occurrence from —OH and optionally substituted —O-C₁-C₈ alkyl, wherein at least one R₁ is an optionally substituted alkyl.

Optional substituents for substituting —O-C₁-C₈ alkyl include C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₃-C₇-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy(C₁-C₆)alkyl (i.e., ether), aryloxy, sulfhydryl (mercapto), halo(C₁-C₆)alkyl (e.g., —CF₃), halo(C₁-C₆)alkoxy (e.g., —OCF₃), C₁-C₆ alkylthio, arylthio, amino, amino(C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O).

In some embodiments, p is 5 (i.e., the cyclodextrin is a β-cyclodextrin). In some embodiments, the alkylated cyclodextrin is a sulfoalkylether-β-cyclodextrin. For example, in some embodiments, at least one R₁ is O-(C₂-C₆ alkylene)-SO₃⁻-T, wherein T is independently selected at each occurrence from pharmaceutically acceptable cations. Suitable examples of T include H⁺, alkali metals (e.g., Li⁺, Na⁺, K³⁰), alkaline earth metals (e.g., Ca⁺², Mg⁺²), ammonium ions and amine cations such as the cations of (C₁-C₆)-alkylamines, piperidine, pyrazine, (C₁-C₆)-alkanolamine, ethylenediamine and (C₄-C₈)-cycloalkanolamine among others, and combinations thereof.

In some embodiments, the alkylated cyclodextrin has the structure of Formula (III):

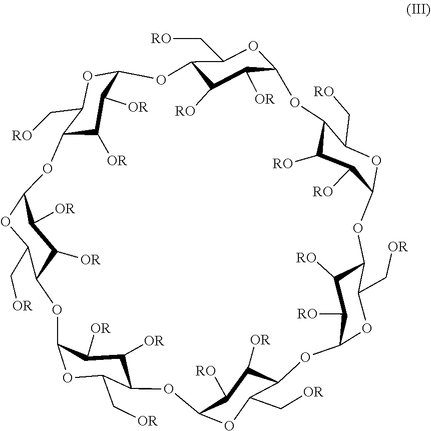

wherein each R is independently —H or —(CH₂)₄—SO₃⁻Na⁺, and the average degree of substitution with —(CH₂)₄—SO₃⁻Na⁺ of all cyclodextrin molecules in the composition is from 6 to 7.1. For example, in some embodiments, the alkylated cyclodextrin may be CAPTISOL®.

In the compositions described herein, individual molecules of cyclodextrin in the composition may have varying degrees of substitution for a specified substituent. Accordingly, it is common to characterize such compositions by an average degree of substitution (ADS) for a specified substituent. Thus, for example, an ADS of 6 to 7.1 indicates that, although each molecule of cyclodextrin in the composition has an integer degree of substitution for a specified substituent, there is a distribution of such degrees of substitution in the composition, resulting in an average of 6 to 7.1.

Further exemplary sulfoalkyl ether (SAE)-CD derivatives include:

TABLE 1

| SAEₓ-α-CD | SAEₓ-β-CD | SAEₓ-γ-CD |
|---|---|---|
| (Sulfoethyl ether)ₓ- | (Sulfoethyl ether)ₓ- | (Sulfoethyl ether)ₓ- |
| α-CD | β-CD | γ-CD |
| (Sulfopropyl ether)ₓ- | (Sulfopropyl ether)ₓ- | (Sulfopropyl ether)ₓ- |
| α-CD | β-CD | γ-CD |
| (Sulfobutyl ether)ₓ- | (Sulfobutyl ether)ₓ- | (Sulfobutyl ether)ₓ- |
| α-CD | β-CD | γ-CD |
| (Sulfopentyl ether)ₓ- | (Sulfopentyl ether)ₓ- | (Sulfopentyl ether)ₓ- |
| α-CD | β-CD | γ-CD |
| (Sulfohexyl ether)ₓ- | (Sulfohexyl ether)ₓ- | (Sulfohexyl ether)ₓ- |
| α-CD | β-CD | γ-CD |

wherein x denotes the average degree of substitution. In some embodiments, the alkylated cyclodextrins are formed as salts.

Various embodiments of a sulfoalkyl ether cyclodextrin include eicosa-O-(methyl)-6G-O-(4-sulfobutyl)-β-cyclodextrin, heptakis-O-(sulfomethyl)-tetradecakis-O-(3-sulfopropyl)-β-cyclodextrin, heptakis-O-[(1,1-dimethylethyl)dimethylsilyl]-tetradecakis-O-(3-sulfopropyl)-β-cyclodextrin, heptakis-O-(sulfomethyl)-tetradecakis-O-(3-sulfopropyl)-β-cyclodextrin, and heptakis-O-[(1,1-dimethylethyl)dimethylsilyl]-tetradecakis-O-(sulfomethyl)-β-cyclodextrin. Other known alkylated cyclodextrins containing a sulfoalkyl moiety include sulfoalkylthio and sulfoalkylthioalkyl ether derivatives such as octakis-(S-sulfopropyl)-octathio-γ-cyclodextrin, octakis-O-[3-[(2-sulfoethyl)thio]propyl]-β-cyclodextrin], and octakis-S-(2-sulfoethyl)-octathio-γ-cyclodextrin.

In some embodiments, an alkylated cyclodextrin composition of the present disclosure is a sulfoalkyl ether-γ-cyclodextrin composition having an ADS of 2 to 9, 4 to 8, 4 to 7.5, 4 to 7, 4 to 6.5, 4.5 to 8, 4.5 to 7.5, 4.5 to 7, 5 to 8, 5 to 7.5, 5 to 7, 5.5 to 8, 5.5 to 7.5, 5.5 to 7, 5.5 to 6.5, 6 to 8, 6 to 7.5, 6 to 7.1, 6.5 to 7.1, 6.2 to 6.9, or 6.5 per alkylated cyclodextrin, and the remaining substituents are —H.

In some embodiments, R¹ of Formula (I) is —OH or unsubstituted —O-C₁-C₈ alkyl. Such alkylated cyclodextrins are known as alkylether (AE)-CDs. Exemplary AE-CD derivatives include:

TABLE 2

| (Alkylether)_{y}-α-CD | (Alkylether)_{y}-β-CD | (Alkylether)_{y}-γ-CD |
|---|---|---|
| ME_{y}-α-CD | ME_{y}-β-CD | ME_{y}-γ-CD |
| EE_{y}-α-CD | EE_{y}-β-CD | EE_{y}-γ-CD |
| PE_{y}-α-CD | PE_{y}-β-CD | PE_{y}-γ-CD |
| BE_{y}-α-CD | BE_{y}-β-CD | BE_{y}-γ-CD |
| PtE_{y}-α-CD | PtE_{y}-β-CD | PtE_{y}-γ-CD |
| HE_{y}-α-CD | HE_{y}-β-CD | HE_{y}-γ-CD |

wherein ME denotes methyl ether, EE denotes ethyl ether, PE denotes propyl ether, BE denotes butyl ether, PtE denotes pentyl ethyl, HE denotes hexyl ether, and y denotes the average degree of substitution.

In some embodiments, at least one R₁ is —O-C₁-C₆ alkyl substituted with hydroxyl (e.g., hydroxypropyl-(3-cyclodextrin). Further exemplary hydroxyalkyl ether (HAE)-CD derivatives include:

TABLE 3

| (HAE)_{z}-α-CD | (HAE)_{z}-β-CD | (HAE)_{z}-γ-CD |
|---|---|---|
| HMEz-α-CD | HMEz-β-CD | HMEz-γ-CD |
| HEEz-α-CD | HEEz-β-CD | HEEz-γ-CD |
| HPEz-α-CD | HPEz-β-CD | HPEz-γ-CD |
| HBEz-α-CD | HBEz-β-CD | HBEz-γ-CD |
| HPtEz-α-CD | HPtEz-β-CD | HPtEz-γ-CD |
| HHEz-α-CD | HHEz-β-CD | HHEz-γ-CD |

wherein HME denotes hydroxymethyl ether, HEE denotes hydroxyethyl ether, HPE denotes hydroxypropyl ether, HBE denotes hydroxybutyl ether, HPtE denotes hydroxypentyl ether, HHE denotes hydroxyhexyl ether, and z denotes the average degree of substitution.

In some embodiments, alkylated cyclodextrins are provided having mixed substituents (e.g., including both sulfoalkylether and alkylether substituents (SAE-AE-CDs)). Specific embodiments of the such derivatives of include those wherein: 1) the alkylene moiety of the SAE has the same number of carbons as the alkyl moiety of the AE; 2) the alkylene moiety of the SAE has a different number of carbons than the alkyl moiety of the AE; 3) the alkyl and alkylene moieties are independently selected from the group consisting of a straight chain or branched moiety; 4) the alkyl and alkylene moieties are independently selected from the group consisting of a saturated or unsaturated moiety; 5) the ADS for the SAE group is greater than or approximates the ADS for the AE group; or 6) the ADS for the SAE group is less than the ADS for the AE group. Some embodiments include a SAE-HAE-CD.

The alkylated cyclodextrin can include SAE-CD, HAE-CD, SAE-HAE-CD, HANE-CD, HAE-AE-CD, HAE-SAE-CD, AE-CD, SAE-AE-CD, neutral cyclodextrin, anionic cyclodextrin, cationic cyclodextrin, halo-derivatized cyclodextrin, amino-derivatized cyclodextrin, nitrile-derivatized cyclodextrin, aldehyde-derivatized cyclodextrin, carboxylate-derivatized cyclodextrin, sulfate-derivatized cyclodextrin, sulfonate-derivatized cyclodextrin, mercapto-derivatized cyclodextrin, alkylamino-derivatized cyclodextrin, or succinyl-derivatized cyclodextrin.

In some embodiments, alkylated cyclodextrins such as mixed ether alkylated cyclodextrins include, by way of example, those listed Table 4 below.

TABLE 4

| Mixed ether | Mixed ether | Mixed ether |
|---|---|---|
| CD derivative | CD derivative | CD derivative |
| Sulfobutyl- | Sulfopropyl- | Sulfoethyl- |
| hydroxybutyl-CD | hydroxybutyl-CD | hydroxybutyl-CD |
| (SBE-HBE-CD) | (SPE-HBE-CD) | (SEE-HBE-CD) |
| Sulfobutyl- | Sulfopropyl- | Sulfoethyl- |
| hydroxypropyl-CD | hydroxypropyl-CD | hydroxypropyl-CD |
| (SBE-HPE-CD) | (SPE-HPE-CD) | (SEE-HPE-CD) |
| Sulfobutyl- | Sulfopropyl- | Sulfoethyl- |
| hydroxyethyl-CD | hydroxyethyl-CD | hydroxyethyl-CD |
| (SBE-HEE-CD) | (SPE-HEE-CD) | (SEE-HEE-CD) |
| Sulfobutyl- | Sulfopropyl- | Sulfoethyl- |
| hydroxybutenyl-CD | hydroxybutenyl-CD | hydroxybutenyl-CD |
| (SBE-HBNE-CD) | (SPE-HBNE-CD) | (SEE-HBNE-CD) |
| Sulfobutyl-ethyl | Sulfopropyl-ethyl | Sulfoethyl-ethyl |
| (SBE-EE-CD) | (SPE-EE-CD) | (SEE-EE-CD) |
| Sulfobutyl-methyl | Sulfopropyl-methyl | Sulfoethyl-methyl |
| (SBE-ME-CD) | (SPE-ME-CD) | (SEE-ME-CD) |
| Sulfobutyl-propyl | Sulfopropyl-propyl | Sulfoethyl-propyl |
| (SBE-PE-CD) | (SPE-PE-CD) | (SEE-PE-CD) |
| Sulfobutyl-butyl | Sulfopropyl-butyl | Sulfoethyl-butyl |
| (SBE-BE-CD) | (SPE-BE-CD) | (SEE-BE-CD) |
| Sulfobutyl- | Sulfopropyl- | Sulfoethyl- |
| carboxymethyl-CD | carboxymethyl-CD | carboxymethyl-CD |
| (SBE-CME-CD) | (SPE-CME-CD) | (SEE-CME-CD) |
| Sulfobutyl- | Sulfopropyl- | Sulfoethyl- |
| carboxyethyl-CD | carboxyethyl-CD | carboxyethyl-CD |
| (SBE-CEE-CD) | (SPE-CEE-CD) | (SEE-CEE-CD) |
| Sulfobutyl- | Sulfopropyl- | Sulfoethyl- |
| acetate-CD | acetate-CD | acetate-CD |
| (SBE-AA-CD) | (SPE-AA-CD) | (SEE-AA-CD) |
| Sulfobutyl- | Sulfopropyl- | Sulfoethyl- |
| propionate-CD | propionate-CD | propionate-CD |
| (SBE-PA-CD) | (SPE-PA-CD) | (SEE-PA-CD) |
| Sulfobutyl- | Sulfopropyl- | Sulfoethyl- |
| butyrate-CD | butyrate-CD | butyrate-CD |
| (SBE-BA-CD) | (SPE-BA-CD) | (SEE-BA-CD) |
| Sulfobutyl- | Sulfopropyl- | Sulfoethyl- |
| methoxycarbonyl-CD | methoxycarbonyl-CD | methoxycarbonyl-CD |
| (SBE-MC-CD) | (SPE-MC-CD) | (SEE-MC-CD) |
| Sulfobutyl- | Sulfopropyl- | Sulfoethyl- |
| ethoxycarbonyl-CD | ethoxycarbonyl-CD | ethoxycarbonyl-CD |
| (SBE-EC-CD) | (SPE-EC-CD) | (SEE-EC-CD) |
| Sulfobutyl- | Sulfopropyl- | Sulfoethyl- |
| propoxycarbonyl-CD | propoxycarbonyl-CD | propoxycarbonyl-CD |
| (SBE-PC-CD) | (SPE-PC-CD) | (SEE-PC-CD) |
| Hydroxybutyl- | Hydroxypropyl- | Hydroxyethyl- |
| hydroxybutenyl-CD | hydroxybutenyl-CD | hydroxybutenyl-CD |
| (HBE-HBNE-CD) | (HPE-HBNE-CD) | (HEE-HBNE-CD) |
| Hydroxybutyl- | Hydroxypropyl- | Hydroxyethyl- |
| ethyl-CD | ethyl-CD | ethyl-CD |
| (HBE-EE-CD) | (HPE-EE-CD) | (HEE-EE-CD) |
| Hydroxybutyl- | Hydroxypropyl- | Hydroxyethyl- |
| methyl-CD | methyl-CD | methyl-CD |
| (HBE-ME-CD) | (HPE-ME-CD) | (HEE-ME-CD) |
| Hydroxybutyl- | Hydroxypropyl- | Hydroxyethyl- |
| propyl-CD | propyl-CD | propyl-Cd |
| (HBE-PE-CD) | (HPE-PE-CD) | (HEE-PE-CD) |
| Hydroxybutyl-butyl | Hydroxypropyl-butyl | Hydroxyethyl-butyl |
| (HBE-BE-CD) | (HPE-BE-CD) | (HEE-BE-CD) |
| Hydroxybutyl- | Hydroxypropyl- | Hydroxyethyl- |
| carboxymethyl-CD | carboxymethyl-CD | carboxymethyl-CD |
| (HBE-CME-CD) | (HPE-CME-CD) | (HEE-CME-CD) |
| Hydroxybutyl- | Hydroxypropyl- | Hydroxyethyl- |
| carboxyethyl-CD | carboxyethyl-CD | carboxyethyl-CD |
| (HBE-CEE-CD) | (HPE-CEE-CD) | (HEE-CEE-CD) |
| Hydroxybutyl- | Hydroxypropyl- | Hydroxyethyl- |
| acetate-CD | acetate-CD | acetate-CD |
| (HBE-AA-CD) | (HPE-AA-CD) | (HEE-AA-CD) |
| Hydroxybutyl- | Hydroxypropyl- | Hydroxyethyl- |
| propionate-CD | propionate-CD | propionate-CD |
| (HBE-PA-CD) | (HPE-PA-CD) | (HEE-PA-CD) |
| Hydroxybutyl- | Hydroxypropyl- | Hydroxyethyl- |
| butyrate-CD | butyrate-CD | butyrate-CD |
| (HBE-BA-CD) | (HPE-BA-CD) | (HEE-BA-CD) |
| Hydroxybutyl- | Hydroxypropyl- | Hydroxyethyl- |
| methoxycarbonyl-CD | methoxycarbonyl-CD | methoxycarbonyl-CD |
| (HBE-MC-CD) | (HPE-MC-CD) | (HEE-MC-CD) |
| Hydroxybutyl- | Hydroxypropyl- | Hydroxyethyl- |
| ethoxycarbonyl-CD | ethoxycarbonyl-CD | ethoxycarbonyl-CD |
| (HBE-EC-CD) | (HPE-EC-CD) | (HEE-EC-CD) |
| Hydroxybutyl- | Hydroxypropyl- | Hydroxyethyl- |
| propoxycarbonyl-CD | propoxycarbonyl-CD | propoxycarbonyl-CD |
| (HBE-PC-CD) | (HPE-PC-CD) | (HEE-PC-CD) |
| Hydroxybutenyl- | Hydroxypropenyl- | Hydroxypentenyl- |
| ethyl-CD | ethyl-CD | ethyl-CD |
| (HBNE-EE-CD) | (HPNE-EE-CD) | (HPTNE-EE-CD) |
| Hydroxybutenyl- | Hydroxypropenyl- | Hydroxypentenyl- |
| methyl-CD | methyl-CD | methyl-CD |
| (HBNE-ME-CD) | (HPNE-ME-CD) | (HPTNE-ME-CD) |
| Hydroxybutenyl- | Hydroxypropenyl- | Hydroxypentenyl- |
| propyl-CD | propyl-CD | propyl-CD |
| (HBNE-PE-CD) | (HPNE-PE-CD) | (HPTNE-PE-CD) |
| Hydroxybutenyl- | Hydroxypropenyl- | Hydroxypentenyl- |
| butyl-CD | butyl-CD | butyl-CD |
| (HBNE-BE-CD) | (HPNE-BE-CD) | (HPTNE-BE-CD) |
| Hydroxybutenyl- | Hydroxypropenyl- | Hydroxypentenyl- |
| carboxymethyl-CD | carboxymethyl-CD | carboxymethyl-CD |
| (HBNE-CME-CD) | (HPNE-CME-CD) | (HPTNE-CME-CD) |
| Hydroxybutenyl- | Hydroxypropenyl- | Hydroxypentenyl- |
| carboxyethyl-CD | carboxyethyl-CD | carboxyethyl-CD |
| (HBNE-CEE-CD)- | (HPNE-CEE-CD) | (HPTNE-CEE-CD) |
| Hydroxybutenyl- | Hydroxypropenyl- | Hydroxypentenyl- |
| acetate-CD | acetate-CD | acetate-CD |
| (HBNE-AA-CD) | (HPNE-AA-CD) | (HPTNE-AA-CD) |
| Hydroxybutenyl- | Hydroxypropenyl- | Hydroxypentenyl- |
| propionate-CD | propionate-CD | propionate-CD |
| (HBNE-PA-CD) | (HPNE-PA-CD) | (HPTNE-PA-CD) |
| Hydroxybutenyl- | Hydroxypropenyl- | Hydroxypentenyl- |
| butyrate-CD | butyrate-CD | butyrate-CD |
| (HBNE-BA-CD) | (HPNE-BA-CD) | (HPTNE-BA-CD) |
| Hydroxybutenyl- | Hydroxypropenyl- | Hydroxypentenyl- |
| methoxycarbonyl-CD | methoxycarbonyl-CD | methoxycarbonyl-CD |
| (HBNE-MC-CD) | (HPNE-MC-CD) | (HPTNE-MC-CD) |
| Hydroxybutenyl- | Hydroxypropenyl- | Hydroxypentenyl- |
| ethoxycarbonyl-CD | ethoxycarbonyl-CD | ethoxycarbonyl-CD |
| (HBNE-EC-CD) | (HPNE-EC-CD) | (HPTNE-EC-CD) |
| Hydroxybutenyl- | Hydroxypropenyl- | Hydroxypentenyl- |
| propoxycarbonyl-CD | propoxycarbonyl-CD | propoxycarbonyl-CD |
| (HBNE-PC-CD) | (HPNE-PC-CD) | (HPTNE-PC-CD) |

Additional examples of alkylated cyclodextrins that may be prepared by the methods disclosed herein are described in U.S. Pat. Nos. 5,438,133, 6,479,467, and 6,610,671, the disclosure of each of which is incorporated by reference herein in its entirety.

Within a given alkylated cyclodextrin composition, the substituents of the alkylated cyclodextrin(s) thereof can be the same or different. For example, SAE or HAE moieties can have the same type or different type of alkylene (alkyl) radical upon each occurrence in an alkylated cyclodextrin composition. In such embodiments, the alkylene radical in the SAE or HAE moiety can be ethyl, propyl, butyl, pentyl or hexyl in each occurrence in an alkylated cyclodextrin composition.

The alkylated cyclodextrins can differ in their degree of substitution by functional groups, the number of carbons in the functional groups, their molecular weight, the number of glucopyranose units contained in the base cyclodextrin used to form the derivatized cyclodextrin and or their substitution patterns. In addition, the derivatization of a cyclodextrin with functional groups occurs in a controlled, although not exact manner. For this reason, the degree of substitution is actually a number representing the average number of functional groups per cyclodextrin (for example, SBE₇-β-CD has an average of 7 substitutions per cyclodextrin). Thus, it has an average degree of substitution (“ADS”) of 7. In some embodiments, the ADS may be determined by techniques include capillary electrophoresis (CE), high performance liquid chromatography (HPLC), nuclear magnetic resonance (NMR) spectroscopy, or a combination thereof. In addition, the regiochemistry of substitution of the hydroxyl groups of the cyclodextrin is variable with regard to the substitution of specific hydroxyl groups of the hexose ring. For this reason, substitution of the different hydroxyl groups is likely to occur during manufacture of the derivatized cyclodextrin, and a particular derivatized cyclodextrin will possess a preferential, although not exclusive or specific, substitution pattern. Given the above, the molecular weight of a particular derivatized cyclodextrin composition can vary from batch to batch.

In a single parent cyclodextrin molecule, there are 3v+6 hydroxyl moieties available for derivatization. Where v=4 (α-cyclodextrin), “y” the degree of substitution for the moiety can range in value from 1 to 18. Where v=5 (β-cyclodextrin), “y” the degree of substitution for the moiety can range in value from 1 to 21. Where v=6 (γ-cyclodextrin), “y” the degree of substitution for the moiety can range in value from 1 to 24. In general, “y” also ranges in value from 1 to 3v+g, where g ranges in value from 0 to 5. In some embodiments, “y” ranges from 1 to 2v+g, or from 1 to 1v+g.

The degree of substitution (“DS”) for a specific moiety (SAE, HAE or AE, for example) is a measure of the number of SAE (HAE or AE) substituents attached to an individual cyclodextrin molecule, in other words, the moles of substituent per mole of cyclodextrin. Therefore, each substituent has its own DS for an individual alkylated cyclodextrin species. The average degree of substitution (“ADS”) for a substituent is a measure of the total number of substituents present per cyclodextrin molecule for the distribution of alkylated cyclodextrins within an alkylated cyclodextrin composition of the present disclosure. Therefore, SAE4-CD has an ADS (per CD molecule) of 4.

Some embodiments of the present disclosure include those wherein: 1) more than half of the hydroxyl moieties of the alkylated cyclodextrin are derivatized; 2) half or less than half of the hydroxyl moieties of the alkylated cyclodextrin are derivatized; 3) the substituents of the alkylated cyclodextrin are the same upon each occurrence; 4) the substituents of the alkylated cyclodextrin comprise at least two different substituents; or 5) the substituents of the alkylated cyclodextrin comprise one or more of substituents selected from the group consisting of unsubstituted alkyl, substituted alkyl, halide (halo), haloalkyl, amine (amino), aminoalkyl, aldehyde, carbonylalkyl, nitrile, cyanoalkyl, sulfoalkyl, hydroxyalkyl, carboxyalkyl, thioalkyl, unsubstituted alkylene, substituted alkylene, aryl, arylalkyl, heteroaryl, and heteroarylalkyl.

Alkylated cyclodextrin compositions can comprise multiple alkylated cyclodextrin molecules differing in degree of substitution. For example, an alkylated cyclodextrin molecule can have 1, 2, 3, 4, 5, 6, 7, 8, 9, 10, 11, 12, or more of the hydroxyl groups of the parent cyclodextrin functionalized with a substituent, e.g., a sulfoalkyl ether. In such compositions, the average degree of substitution (ADS) can be calculated, as described herein, based on the relative amounts of alkylated cyclodextrin molecules having a particular degree of substitution. As a consequence, the ADS for SAE of a SAE-CD derivative composition represents a weighted average of the degree of substitution of the individual SAE-CD molecules in the composition. For example, a SAE_{5.2}-CD composition comprises a distribution of multiple SAEₓ-CD molecules, wherein “x” (the DS for SAE groups) can range from integers having values of 1 to 12 for individual cyclodextrin molecules; however, the population of SAE-cyclodextrin molecules is such that the average value for “x” (the ADS for SAE groups) is 5.2.

The alkylated cyclodextrin compositions can have a high to moderate to low ADS. The alkylated cyclodextrin compositions can also have a wide or narrow “span,” which is the number of alkylated cyclodextrin molecules with differing degrees of substitution within an alkylated cyclodextrin composition. For example, an alkylated cyclodextrin composition comprising a single species of alkylated cyclodextrin having a single degree of substitution is said to have a span of one, and in such a case, the degree of substitution for the alkylated cyclodextrin molecule would equal the ADS of its alkylated cyclodextrin composition. An electropherogram, for example, of an alkylated cyclodextrin with a span of one should have only one alkylated cyclodextrin species with respect to degree of substitution. An alkylated cyclodextrin composition having a span of two comprises two individual alkylated cyclodextrin species differing in their degree of substitution, and its electropherogram, for example, would indicate two different alkylated cyclodextrin species differing in degree of substitution. Likewise, the span of an alkylated cyclodextrin composition having a span of three comprises three individual alkylated cyclodextrin species differing in their degree of substitution. The span of an alkylated cyclodextrin composition typically ranges from 5 to 15, or 7 to 12, or 8 to 11.

A parent cyclodextrin includes a secondary hydroxyl group on the C-2 and C-3 positions of the glucopyranose residues forming the cyclodextrin and a primary hydroxyl on the C-6 position of the same. Each of these hydroxyl moieties is available for derivatization by substituent precursor. Depending upon the synthetic methodology employed, the substituent moieties can be distributed randomly or in a somewhat ordered manner among the available hydroxyl positions. The regioisomerism of derivatization by the substituent can also be varied as desired. The regioisomerism of each composition is independently selected. For example, a majority of the substituents present can be located at a primary hydroxyl group or at one or both of the secondary hydroxyl groups of the parent cyclodextrin. In some embodiments, the primary distribution of substituents is C-3>C-2>C-6, while in other embodiments the primary distribution of substituents is C-2>C-3>C-6. Some embodiments of the present disclosure include an alkylated cyclodextrin molecule wherein a minority of the substituent moieties is located at the C-6 position, and a majority of the substituent moieties is located at the C-2 and/or C-3 position. Still other embodiments of the present disclosure include an alkylated cyclodextrin molecule wherein the substituent moieties are substantially evenly distributed among the C-2, C-3, and C-6 positions.

An alkylated cyclodextrin composition comprises a distribution of plural individual alkylated cyclodextrin species, each species having an individual degree of substitution (“IDS”). The content of each of the cyclodextrin species in a particular composition can be quantified using capillary electrophoresis. The method of analysis (capillary electrophoresis, for example, for charged alkylated cyclodextrins) is sufficiently sensitive to distinguish between compositions having only 5% of one alkylated cyclodextrin and 95% of another alkylated cyclodextrin from starting alkylated cyclodextrin compositions containing a single alkylated cyclodextrin.

The above-mentioned variations among the individual species of alkylated cyclodextrins in a distribution can lead to changes in the complexation equilibrium constant K_{1:1} which in turn will affect the required molar ratios of the derivatized cyclodextrin to active agent. The equilibrium constant is also somewhat variable with temperature and allowances in the ratio are required such that the agent remains solubilized during the temperature fluctuations that can occur during manufacture, storage, transport, and use. The equilibrium constant can also vary with pH and allowances in the ratio can be required such that the agent remains solubilized during pH fluctuations that can occur during manufacture, storage, transport, and use. The equilibrium constant can also vary due the presence of other excipients (e.g., buffers, preservatives, antioxidants). Accordingly, the ratio of derivatized cyclodextrin to active agent can be varied from the ratios set forth herein in order to compensate for the above-mentioned variables.

### Pharmaceutical Compositions

In some embodiments, the pharmaceutical compositions described herein contain a compound of Formula (I) and an alkylated cyclodextrin in a pre-determined molar ratio. In various embodiments, the molar ratio of alkylated cyclodextrin to compound of Formula (I) has a lower limit of 1:1, 2:1, 3:1, 4:1, 5:1, 6:1, 7:1, 8:1, 9:1, 10:1, 15:1, 20:1, 30:1, 50:1, or 100:1 and an upper limit of 10:1, 15:1, 20:1, 30:1, 40:1, 50:1, 70:1, 100:1, 120:1, 150:1, 200:1, 250:1, 300:1, 350:1, 400:1, 450:1, 500:1, 600:1, 750:1, and 1000:1. For example, in various embodiments, the molar ratio of alkylated cyclodextrin to compound of Formula (I) is from 1:1 to 500:1, 1:1 to 300:1, 1:1 to 150:1, 1:1 to 100:1, 2:1 to 350:1, 2:1 to 200:1, 2:1 to 100:1, 3:1 to 200:1, 3:1 to 150:1, 3:1, to 100:1, 3:1 to 50:1, 4:1 to 150:1, and 4:1 to 100:1.

The pharmaceutical composition may be provided as a solid or a liquid formulation. For example, a solid formulation may be provided that is suitable for reconstitution using a diluent prior to administration to a subject. Suitable diluents for reconstitution can include, for example, sterile water or saline solution. When provided as a liquid formulation, or upon reconstitution of a solid formulation, the composition may be an aqueous solution or suspension.

A liquid formulation of the disclosure can be converted to a solid formulation for reconstitution. A reconstitutable solid composition according to the disclosure comprises an active agent, a derivatized cyclodextrin and optionally at least one other pharmaceutical excipient. A reconstitutable composition can be reconstituted with an aqueous liquid to form a liquid formulation that is preserved. The composition can comprise an admixture (minimal to no presence of an inclusion complex) of a solid derivatized cyclodextrin and an active agent-containing solid and optionally at least one solid pharmaceutical excipient, such that a major portion of the active agent is not complexed with the derivatized cyclodextrin prior to reconstitution. Alternatively, the composition can comprise a solid mixture of a derivatized cyclodextrin and an active agent, wherein a major portion of the active agent is complexed with the derivatized cyclodextrin prior to reconstitution. A reconstitutable solid composition can also comprise a derivatized cyclodextrin and an active agent where substantially all or at least a major portion of the active agent is complexed with the derivatized cyclodextrin.

A reconstitutable solid composition can be prepared according to any of the following processes. A liquid formulation of the disclosure is first prepared, then a solid is formed by lyophilization (freeze-drying), spray-drying, spray freeze-drying, antisolvent precipitation, aseptic spray drying, various processes utilizing supercritical or near supercritical fluids, or other methods known to those of ordinary skill in the art to make a solid for reconstitution.

A liquid vehicle included in a formulation of the disclosure can comprise an aqueous liquid carrier (e.g., water), an aqueous alcohol, an aqueous organic solvent, a non-aqueous liquid carrier, and combinations thereof.

The composition of the present disclosure can include one or more pharmaceutical excipients such as a conventional preservative, antifoaming agent, antioxidant, buffering agent, acidifying agent, alkalizing agent, complexation-enhancing agent, cryoprotectant, electrolyte, glucose, emulsifying agent, oil, plasticizer, solubility-enhancing agent, stabilizer, tonicity modifier, diluent, complexing agents, other excipients known by those of ordinary skill in the art for use in formulations, combinations thereof.

As used herein, the term “alkalizing agent” is intended to mean a compound used to provide alkaline medium for product stability. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids and trolamine and others known to those of ordinary skill in the art.

As used herein, the term “acidifying agent” is intended to mean a compound used to provide an acidic medium for product stability. Such compounds include, by way of example and without limitation, acetic acid, acidic amino acids, citric acid, fumaric acid and other α-hydroxy acids, hydrochloric acid, ascorbic acid, phosphoric acid, sulfuric acid, tartaric acid and nitric acid and others known to those of ordinary skill in the art.

As used herein, a conventional preservative is a compound used to at least reduce the rate at which bioburden increases, but maintains bioburden steady or reduces bioburden after contamination. Such compounds include, by way of example and without limitation, benzalkonium chloride, benzethonium chloride, benzoic acid, benzyl alcohol, cetylpyridinium chloride, chlorobutanol, phenol, phenylethyl alcohol, phenylmercuric nitrate, phenylmercuric acetate, thimerosal, metacresol, myristylgamma picolinium chloride, potassium benzoate, potassium sorbate, sodium benzoate, sodium propionate, sorbic acid, thymol, and methyl, ethyl, propyl or butyl parabens and others known to those of ordinary skill in the art. It is understood that some preservatives can interact with the alkylated cyclodextrin thus reducing the preservative effectiveness. Nevertheless, by adjusting the choice of preservative and the concentrations of preservative and the alkylated cyclodextrin adequately preserved formulations can be found.

As used herein, the term “antioxidant” is intended to mean an agent that inhibits oxidation and thus is used to prevent the deterioration of preparations by the oxidative process. Such compounds include, by way of example and without limitation, acetone, potassium metabisulfite, potassium sulfite, ascorbic acid, ascorbyl palmitate, citric acid, butylated hydroxyanisole, butylated hydroxytoluene, hypophophorous acid, monothioglycerol, propyl gallate, sodium ascorbate, sodium citrate, sodium sulfide, sodium sulfite, sodium bisulfite, sodium formaldehyde sulfoxylate, thioglycolic acid, EDTA, pentetate, and sodium metabisulfite and others known to those of ordinary skill in the art.

As used herein, the term “buffering agent” is intended to mean a compound used to resist change in pH upon dilution or addition of acid or alkali. Such compounds include, by way of example and without limitation, acetic acid, sodium acetate, adipic acid, benzoic acid, sodium benzoate, boric acid, sodium borate, citric acid, glycine, maleic acid, monobasic sodium phosphate, dibasic sodium phosphate, 4-(2-hydroxyethyl)-1-piperazineethanesulfonic acid, lactic acid, tartaric acid, potassium metaphosphate, potassium phosphate, monobasic sodium acetate, sodium bicarbonate, tris, sodium tartrate and sodium citrate anhydrous and dihydrate and others known to those of ordinary skill in the art.

A complexation-enhancing agent can be added to a formulation of the disclosure. When such an agent is present, the ratio of cyclodextrin/active agent can be changed. A complexation-enhancing agent is a compound, or compounds, that enhance(s) the complexation of the active agent with the cyclodextrin. Suitable complexation enhancing agents include one or more pharmacologically inert water-soluble polymers, hydroxy acids, and other organic compounds typically used in preserved formulations to enhance the complexation of a particular agent with cyclodextrins.

Hydrophilic polymers can be used as complexation-enhancing, solubility-enhancing and/or water activity reducing agents to improve the performance of formulations containing a CD-based preservative. Loftsson has disclosed a number of polymers suitable for combined use with a cyclodextrin (underivatized or derivatized) to enhance the performance and/or properties of the cyclodextrin. Suitable polymers are disclosed in Pharmazie 56:746 (2001); Int. J. Pharm. 212:29 (2001); Cyclodextrin: From Basic Research to Market, 10th Int'l Cyclodextrin Symposium, Ann Arbor, MI, US, May 21-24, p. 10-15 (2000); PCT Int'l Pub. No. WO 99/42111; Pharmazie 53:733 (1998); Pharm. Technol. Eur. 9:26 (1997); J. Pharm. Sci. 85:1017 (1996); European Patent Appl. No. 0 579 435; Proc. of the 9th Int'l Symposium on Cyclodextrins, Santiago de Comostela, ES, May 31-Jun. 3, 1998, pp. 261-264 (1999); S.T.P. Pharma Sciences 9:237 (1999); Amer. Chem. Soc. Symposium Series 737 (Polysaccharide Applications):24-45 (1999); Pharma. Res. 15:1696 (1998); Drug Dev. Ind. Pharm. 24:365 (1998); Int. J. Pharm. 163:115 (1998); Book of Abstracts, 216th Amer. Chem. Soc. Nat'l Meeting, Boston, Aug. 23-27 CELL-016 (1998); J. Controlled Release 44:95 (1997); Pharm. Res. (1997) 14(11), S203; Invest. Ophthalmol. Vis. Sci. 37:1199 (1996); Proc. of the 23rd Int'l Symposium on Controlled Release of Bioactive Materials 453-454 (1996); Drug Dev. Ind. Pharm. 22:401 (1996); Proc. of the 8th Int'l Symposium on Cyclodextrins, Budapest, HU, Mar. 31-Apr. 2, 1996, pp. 373-376 (1996); Pharma. Sci. 2:277 (1996); Eur. J. Pharm. Sci. 4S:S144 (1996); 3rd Eur. Congress of Pharma. Sci. Edinburgh, Scotland, UK Sep. 15-17, 1996; Pharmazie 51:39 (1996); Eur. J. Pharm. Sci. 4S:S143 (1996); U.S. Pat. Nos. 5,472,954 and 5,324,718; Int. J. Pharm. 126:73 (1995); Abstracts of Papers of the Amer. Chem. Soc. 209:33-CELL (1995); Eur. J. Pharm. Sci. 2:297 (1994); Pharm. Res. 11:S225 (1994); Int. J. Pharm. 104:181 (1994); and Int. J. Pharm. 110:169 (1994), the entire disclosures of which are hereby incorporated by reference in their entirety.

Other suitable polymers are well-known excipients commonly used in the field of pharmaceutical formulations and are included in, for example, Remington's Pharmaceutical Sciences, 18th ed., pp. 291-294, A. R. Gennaro (editor), Mack Publishing Co., Easton, PA (1990); A. Martin et al., Physical Pharmacy. Physical Chemical Principles in Pharmaceutical Sciences, 3d ed., pp. 592-638 (Lea & Febinger, Philadelphia, PA (1983); A. T. Florence et al., Physicochemical Principles of Pharmacy, 2d ed., pp. 281-334, MacMillan Press, London, UK (1988), the disclosures of which are incorporated herein by reference in their entirety. Still other suitable polymers include water-soluble natural polymers, water-soluble semi-synthetic polymers (such as the water-soluble derivatives of cellulose) and water-soluble synthetic polymers. The natural polymers include polysaccharides such as inulin, pectin, algin derivatives (e.g. sodium alginate) and agar, and polypeptides such as casein and gelatin. The semi-synthetic polymers include cellulose derivatives such as methylcellulose, hydroxyethylcellulose, hydroxypropylcellulose, their mixed ethers such as hydroxypropylmethylcellulose and other mixed ethers such as hydroxyethyl-ethylcellulose and hydroxypropylethylcellulose, hydroxypropylmethylcellulose phthalate and carboxymethylcellulose and its salts, especially sodium carboxymethylcellulose. The synthetic polymers include polyoxyethylene derivatives (polyethylene glycols) and polyvinyl derivatives (polyvinyl alcohol, polyvinylpyrrolidone and polystyrene sulfonate) and various copolymers of acrylic acid (e.g. carbomer). Other natural, semi-synthetic and synthetic polymers not named here which meet the criteria of water solubility, pharmaceutical acceptability and pharmacological inactivity are likewise considered to be within the ambit of the present disclosure.

As used herein, the term “stabilizer” is intended to mean a compound used to stabilize the therapeutic agent against physical, chemical, or biochemical process which would reduce the therapeutic activity of the agent. Suitable stabilizers include, by way of example and without limitation, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and other known to those of ordinary skill in the art.

As used herein, the term “tonicity modifier” is intended to mean a compound or compounds that can be used to adjust the tonicity of the liquid formulation. Suitable tonicity modifiers include glycerin, lactose, mannitol, dextrose, sodium chloride, sodium sulfate, sorbitol, trehalose and others known to those of ordinary skill in the art. In some embodiments, the tonicity of the liquid formulation approximates the tonicity of blood or plasma.

As used herein, the term “antifoaming agent” is intended to mean a compound or compounds that prevents or reduces the amount of foaming that forms on the surface of the liquid formulation. Suitable antifoaming agents include dimethicone, simethicone, octoxynol and others known to those of ordinary skill in the art.

As used herein, the term “cryoprotectant” is intended to mean a compound used to protect an active therapeutic agent from physical or chemical degradation during lyophilization. Such compounds include, by way of example and without limitation, dimethyl sulfoxide, glycerol, trehalose, propylene glycol, polyethylene glycol, and others known to those of ordinary skill in the art.

As used herein, the term “emulsifier” or “emulsifying agent” is intended to mean a compound added to one or more of the phase components of an emulsion for the purpose of stabilizing the droplets of the internal phase within the external phase. Such compounds include, by way of example and without limitation, lecithin, polyoxylethylene-polyoxypropylene ethers, polyoxylethylene-sorbitan monolaurate, polysorbates, sorbitan esters, stearyl alcohol, tyloxapol, tragacanth, xanthan gum, acacia, agar, alginic acid, sodium alginate, bentonite, carbomer, sodium carboxymethylcellulose, cholesterol, gelatin, hydroxyethyl cellulose, hydroxypropyl cellulose, octoxynol, oleyl alcohol, polyvinyl alcohol, povidone, propylene glycol monostearate, sodium lauryl sulfate, and others known to those of ordinary skill in the art.

A solubility-enhancing agent can be added to the formulation of the present disclosure. A solubility-enhancing agent is a compound, or compounds, that enhance(s) the solubility of the active agent when in a liquid formulation. When such an agent is present, the ratio of cyclodextrin/active agent can be changed. Suitable solubility enhancing agents include one or more organic solvents, detergents, soaps, surfactant and other organic compounds typically used in parenteral formulations to enhance the solubility of a particular agent.

Suitable organic solvents include, for example, ethanol, glycerin, polyethylene glycols, propylene glycol, poloxomers, and others known to those of ordinary skill in the art.

Formulations comprising the alkylated cyclodextrin of the present disclosure can include oils (e.g., fixed oils, peanut oil, sesame oil, cottonseed oil, corn oil olive oil, and the like), fatty acids (e.g., oleic acid, stearic acid, isostearic acid, and the like), fatty acid esters (e.g., ethyl oleate, isopropyl myristate, and the like), fatty acid glycerides, acetylated fatty acid glycerides, and combinations thereof. Formulations comprising the alkylated cyclodextrin of the present disclosure can also include alcohols (e.g., ethanol, iso-propanol, hexadecyl alcohol, glycerol, propylene glycol, and the like), glycerol ketals (e.g., 2,2-dimethyl-1,3-dioxolane-4-methanol, and the like), ethers (e.g., poly(ethylene glycol) 450, and the like), petroleum hydrocarbons (e.g., mineral oil, petrolatum, and the like), water, surfactants, suspending agents, emulsifying agents, and combinations thereof

When provided as an aqueous solution, compositions described herein may contain compounds of Formula (I) at a variety of concentrations, including but not limited to concentration ranges having a lower limit of 25 μM, 30 μM, 40 μM, 50 μM, 60 μM, 70 μM, 80 μM, 90 μM, 100 μM, 110 μM, 120 μM, 150 μM, or 200 μM and an upper limit of 500 μM, 400 μM, 300 μM, 250 μM, 225 μM, 200 μM, 175 μM, 150 μM, 125 μM, and 100 μM. For example, in various embodiments, the concentration of the compound of Formula (I) is from 50 μM to 200 μM, from 75 μM to 150 μM, from 80 μM to 120 μM, from 90 μM to 110 μM, or about 100 μM.

In some embodiments, liquid formulations described herein include buffers and/or tonicity modifying agents to achieve properties suitable for injection into the eye. For example, in various embodiments, the pH of such liquid compositions may range from 6.0 to 8.0, 6.0 to 7.5, 6.0 to 6.8, 6.5 to 7.5, or 6.8 to 7.2. In various embodiments, the osmolality of such liquid compositions may range from 200 mOsm to 500 mOsm, 200 mOsm to 400 mOsm, or from 250 mOsm to 350 mOsm.

While not being bound by any particular theory, it is believed that the trans form of compounds of Formula (I) have a higher binding affinity with alkylated cyclodextrins than the cis form. Furthermore, while the guest-host complex of compounds of Formula (I) with alkylated cyclodextrin enhances the solubility of the compounds, it is believed to also decrease the availability of the compounds for biological activity. Accordingly, a balance is desired between solubility enhancement and bioavailability. In order to achieve this balance, the ratio of cyclodextrin to compound can be selected as described above. In addition, the relative amounts of cis and trans forms of compounds of Formula (I) may also be selected to achieve desired percentages of compound complexed with cyclodextrin and free.

In various embodiments of the compositions described herein, the compounds of formula (I) may be completely or substantially completely in a cis or trans configuration. In various embodiments, greater than 50%, 55%, 60%, 65%, 70%, 75%, 80%, 85%, 90%, 95%, 98%, 99%, 99.5%, or 99.9% of the molecules of Formula (I) in the composition are in the trans configuration. In various other embodiments, greater than 50%, 55%, 60%, 65%, 70%, 75%, 80%, 85%, 90%, 95%, 98%, 99%, 99.5%, or 99.9% of the molecules of Formula (I) in the composition are in the cis configuration.

While not being bound by any particular theory, the trans form of compounds of Formula (I) are the most stable thermodynamically. Accordingly, in some embodiments, a composition is provided that has greater than 50% of compounds in the trans form. However, the compounds of Formula (I) may undergo stereoisomerization upon exposure to various wavelengths of light. Accordingly, in some embodiments, prior to administration of a composition, the relative amounts of cis and trans forms may be altered by exposing the composition to light. Thus, in some embodiments, a composition is provided having greater than 50% of the molecules of Formula (I) in the trans configuration, which is then exposed to light to convert the composition into one having greater than 50% of the molecules of Formula (I) in the cis configuration, or at least a greater amount of compound in the cis configuration than prior to exposure to light. Thus, a composition may be provided having high stability prior to administration, which is then converted to a composition having high bioavailability immediately prior to administration. In some embodiments, the light is UV light.

In some embodiments, the composition is maintained in a light filtering or light blocking container during storage (e.g., an amber vial), and then transferred to a clear container for a certain period of time prior to administration. In some embodiments, the composition is specifically exposed to light of appropriate wavelength, intensity, and duration to achieve the desired level of conversion. In various embodiments, the light provided includes light having wavelengths between 350 nm and 500 nm, between 350 nm and 450 nm, between 350 nm and 400 nm, about 360 nm, about 380 nm, or about 450 nm. In some embodiments, the light can be narrow-spectrum, but in others has a broad spectrum so long as the desired wavelengths are included in the light spectrum. In various embodiments, the total amount of light provided ranges from 100 J/ml to 5000 J/ml, 150 J/ml to 4000 J/ml, 200 J/ml to 2000 J/ml, or 400 J/ml to 1000 J/ml. In some embodiments, total amount of light provided within a specified wavelength ranges from 100 J/ml to 5000 J/ml, 150 J/ml to 4000 J/ml, 200 J/ml to 2000 J/ml, or 400 J/ml to 1000 J/ml. In some embodiments, exposure to light may occur after administration of the composition into the eye.

In some embodiments, compositions are provided having greater than 50%, 60%, 70%, 80%, 90%, 95%, 99%, or 99.5% of the compound of Formula (I) complexed with the cyclodextrin, with the remaining amount of compound in free, un-complexed form. In some embodiments, compositions are provided having less than 90%, 80%, 70%, 60%, 50%, 40%, 30%, 20%, 10%, or 5% of the compound complexed with the cyclodextrin. In some embodiments, compositions are provided having a first percentage of compound of Formula (I) complexed with the cyclodextrin, which is then exposed to light to generate a composition having a second, lower percentage of compound of Formula (I) complexed with the cyclodextrin.

Methods for preparing a liquid formulation disclosed herein can include a first method comprising: forming a first aqueous solution comprising an alkylated cyclodextrin composition; forming a second solution or suspension comprising one or more active agents; and mixing the first and second solutions to form a liquid formulation. A similar second method comprises adding one or more active agents directly to a first solution without formation of the second solution. A third method comprises adding an alkylated cyclodextrin directly to a solution/suspension containing one or more active agents. A fourth method comprises adding a solution comprising one or more active agents to a powdered or particulate alkylated cyclodextrin composition. A fifth method comprises adding one or more active agents directly to a powdered or particulate alkylated cyclodextrin composition, and adding the resulting mixture to a second solution. A sixth method comprises creating a liquid formulation by any of the above methods and then isolating a solid material by lyophilization, spray-drying, aseptic spray drying, spray-freeze-drying, antisolvent precipitation, a process utilizing a supercritical or near supercritical fluid, or another method known to those of ordinary skill in the art to make a powder for reconstitution.

Specific embodiments of the methods of preparing a liquid formulation include those wherein: 1) the method further comprises sterile filtering the formulation using a filtration medium having a pore size of 0.1 μm or larger; 2) the liquid formulation is sterilized by irradiation or autoclaving; 3) the method further comprises isolating a solid from the solution; 4) the solution is purged with nitrogen or argon or other inert pharmaceutically acceptable gas such that a substantial portion of the oxygen dissolved in, and/or in surface contact with, the solution is removed.

Some embodiments of a reconstitutable solid pharmaceutical composition includes those wherein: 1) the pharmaceutical composition comprises an admixture of an alkylated cyclodextrin composition and a solid comprising one or more active agents and optionally at least one solid pharmaceutical excipient, such that a major portion of the active agent is not complexed with an alkylated cyclodextrin prior to reconstitution; and/or 2) the composition comprises a solid mixture of an alkylated cyclodextrin composition and one or more active agents, wherein a major portion of the one or more active agents is complexed with the alkylated cyclodextrin prior to reconstitution.

### Methods of Use

Compositions described herein can be used to treat various retinal disorders. For example, in some embodiments, compositions described herein can be used to restore light sensitivity to a retina, or confer light sensitivity to a cell in the eye. For example, the compositions described herein can confer light sensitivity to retinal pigment epithelial cells and cells disposed in the neurosensory retina, for example, photoreceptor cells and Mueller cells.

Exemplary conditions which are amenable to treatment with the compositions described herein include, but are not necessarily limited to, diabetic retinopathy, age-related macular degeneration (AMD or ARMD) (wet form); dry AMD; retinopathy of prematurity; retinitis pigmentosa (RP); diabetic retinopathy; and glaucoma, including open-angle glaucoma (e.g., primary open-angle glaucoma), angle-closure glaucoma, and secondary glaucomas (e.g., pigmentary glaucoma, pseudoexfoliative glaucoma, and glaucomas resulting from trauma and inflammatory diseases).

Further exemplary conditions amenable to treatment with the compositions disclosed herein include, but are not necessarily limited to, retinal detachment, age-related or other maculopathies, photic retinopathies, surgery-induced retinopathies, toxic retinopathies, retinopathy of prematurity, retinopathies due to trauma or penetrating lesions of the eye, inherited retinal degenerations, surgery-induced retinopathies, toxic retinopathies, retinopathies due to trauma or penetrating lesions of the eye.

Specific exemplary inherited conditions suitable for treatment with the compositions disclosed herein include, but are not necessarily limited to, Bardet-Biedl syndrome (autosomal recessive); Congenital amaurosis (autosomal recessive); Cone or cone-rod dystrophy (autosomal dominant and X-linked forms); Congenital stationary night blindness (autosomal dominant, autosomal recessive and X-linked forms); Macular degeneration (autosomal dominant and autosomal recessive forms); Optic atrophy, autosomal dominant and X-linked forms); Retinitis pigmentosa (autosomal dominant, autosomal recessive and X-linked forms); Syndromic or systemic retinopathy (autosomal dominant, autosomal recessive and X-linked forms); and Usher syndrome (autosomal recessive).

Compositions described herein can be delivered to the eye through a variety of routes. A subject pharmaceutical composition may be delivered intraocularly, by topical application to the eye or by intraocular injection into, for example the vitreous or subretinal (interphotoreceptor) space. Alternatively, a subject pharmaceutical composition may be delivered locally by insertion or injection into the tissue surrounding the eye. A subject pharmaceutical composition may be delivered systemically through an oral route or by subcutaneous, intravenous or intramuscular injection. Alternatively, a subject pharmaceutical composition may be delivered by means of a catheter or by means of an implant, wherein such an implant is made of a porous, non-porous or gelatinous material, including membranes such as silastic membranes or fibers, biodegradable polymers, or proteinaceous material. A subject pharmaceutical composition can be administered prior to the onset of the condition, to prevent its occurrence, for example, during surgery on the eye, or immediately after the onset of the pathological condition or during the occurrence of an acute or protracted condition.

### EXAMPLES

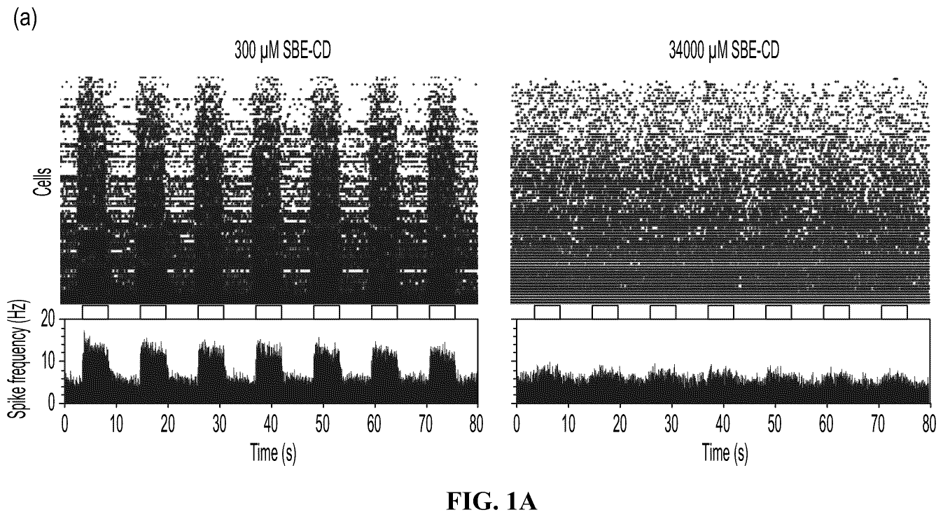

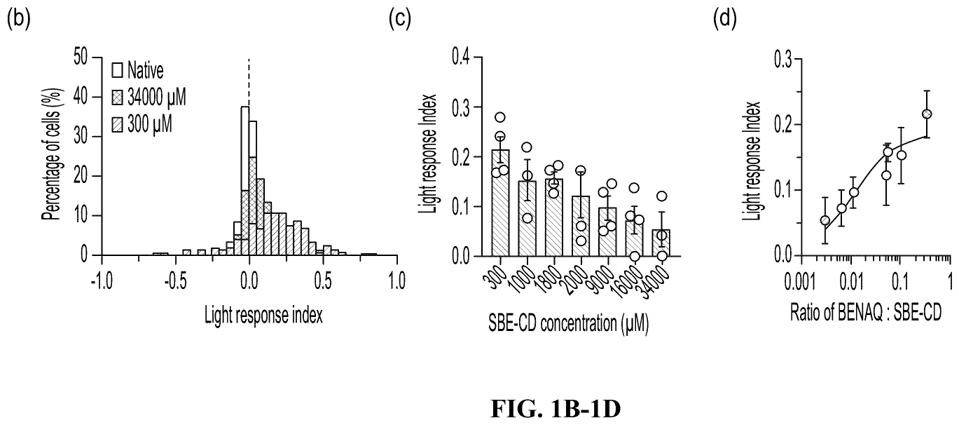

Isolated rd1 retinas were treated with formulations of 100 μM Compound 1 and increasing concentrations of SBE-CD, from 300 μM to 34000 μM. Light responsivity was measured using multielectrode array (MEA) electrophysiology. FIG. 1a shows representative spike rasters and firing frequency plots of retinal ganglion cells (RGCs) from rd1 retina treated with 100 μM Compound 1 complexed with either 300 μM or 34000 μM SBE-CD. Using the minimal amount of SBE-CD to keep Compound 1 fully soluble in the bath solution (300 μM), achieved the strongest light-responses whereas using an excess of SBE-CD (34000 μM) nearly eliminated any light response (FIG. 1a). To quantify the degree of light-responsivity for each recorded cell, the normalized relative difference in observed spike frequencies with and without light stimulus was defined as the light response index (LRI):

$$LRI = \text{?}$$

$$\text{?} \text{indicates text missing or illegible when filed}$$

Histograms generated using the LRI showed that compared to untreated rd1 tissue, an excess of SBE-CD could still allow Compound 1 to photosensitize the cells, albeit very weakly. In contrast, a minimal amount of SBE-CD greatly increased the number of light-responding RGCs (FIG. 1b and 1c). Furthermore, the LRI was found to have a dose-response to the amount of SBE-CD used, suggesting that the efficacy of Compound 1 can be tuned depending on the stoichiometric ratio between it and SBE-CD (FIG. 1d).

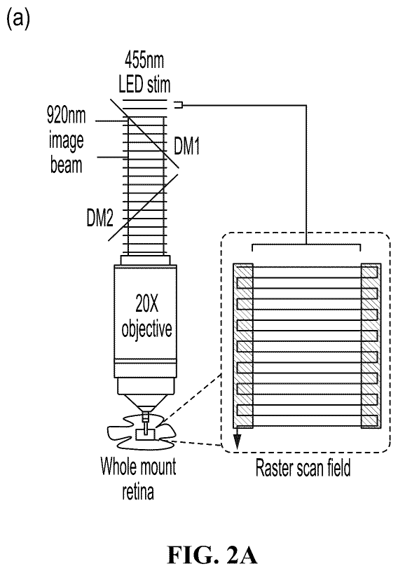

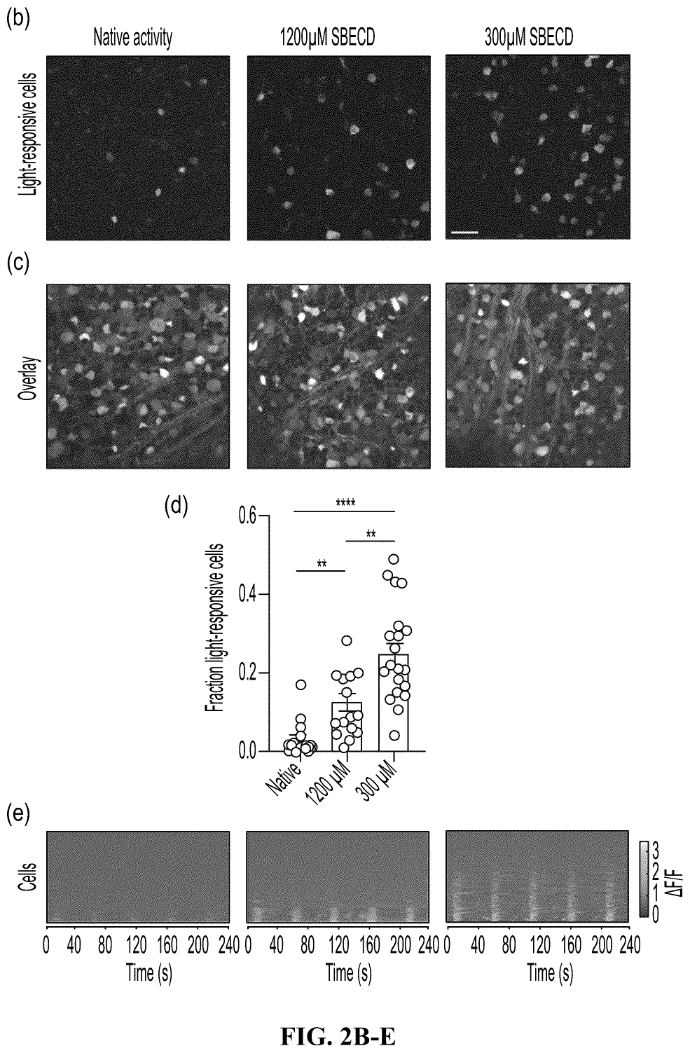

To better understand how Compound 1 was photosensitizing RGCs, an rd1 mouse line was generated expressing the genetically encoded calcium indicator GCalVIP6f under control of Synapsin-1 to label all neurons for 2-photon live imaging. Isolated retina from these mice were then treated with 100 uM Compound 1 and either 300 uM or 1200 uM SBE-CD and imaged in the presence of synaptic blockers. The latter concentration was chosen to increase the amount of available cyclodextrin around to potentiate the photosensitization while not completely quenching it. Due to the spectral overlap of Compound 1 absorbance with GCaMP6fref, an interline stimulus module was developed for the 2-photon microscope. A 455 nm stimulus beam was coupled into the 2-photon imaging beam path under control of a MHz driver that would pulse a wide-field flash of stimulus light in between lines of the raster scan field (FIG. 2a). This setup could then capture live movies of the retina while simultaneously photostimulating Compound 1-treated RGCs.

To determine which neurons were responding to light, collected movies were cross correlated to the stimulus periods using an autocorrelation estimation. Positively correlated pixels, e.g. pixels that showed an increase in intensity during the stimulus period, were highlighted in green and negatively correlated pixels in magenta. These resulting correlated micrographs were then used to quantify the number of light-responsive cells. FIG. 2b shows representative correlation micrographs from rd1-GCaMP6f retina treated 100 μM Compound 1 and either 1200 μM or 300 μM SBE-CD. Cross correlation of GCalVIP6f activity to the stimulus was used to identify light-responsive pixels, shown in green. Pixels with negative correlation are shown in magenta (scale bar=30 μm).

The total number of cells were counted from a max Z-projection the same movies and used to determine the relative fraction of light-responsive cells per Compound 1 formulation. FIG. 2c shows overlays of correlation micrographs with max Z-projections of the original GCaMP6f movies. FIG. 2d shows the fraction of light-responding cells from rd1-GCaMP6f retina treated with the two preparations of Compound 1 and SBE-CD, compared to an untreated retina (P>0.01=**, P>0.0001=****). A lower stoichiometry of SBE-CD resulted in a greater number of RGCs that were light responsive. Also observed was an intrinsic light response in untreated rd1 tissue, where light-responsive cells accounted for approximately 2% of the total number of cells, suggesting that they may be melanopsin containing ipRGCs. These images reveal that the responsive cells are distributed throughout the tissue and are larger in size than the intrinsic light-responsive cells. The activity was also rastered using the recorded ΔF/F of all neurons (FIG. 2e).

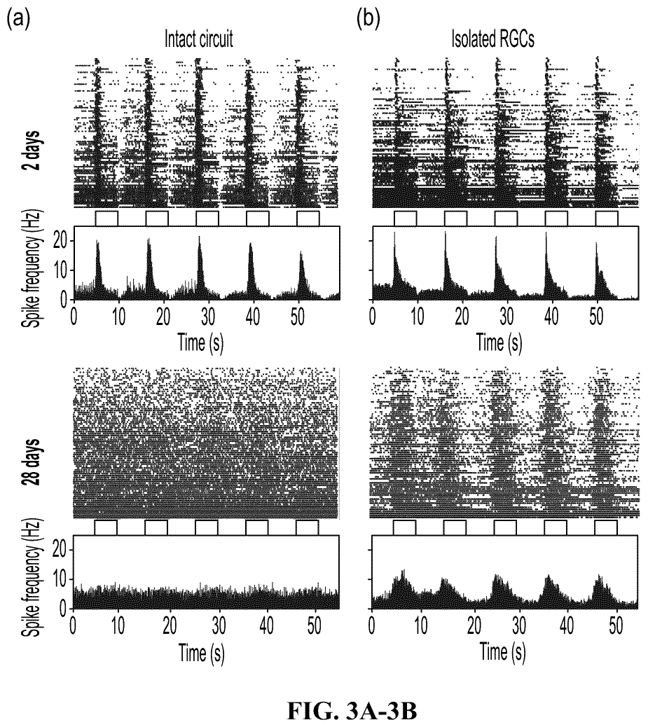

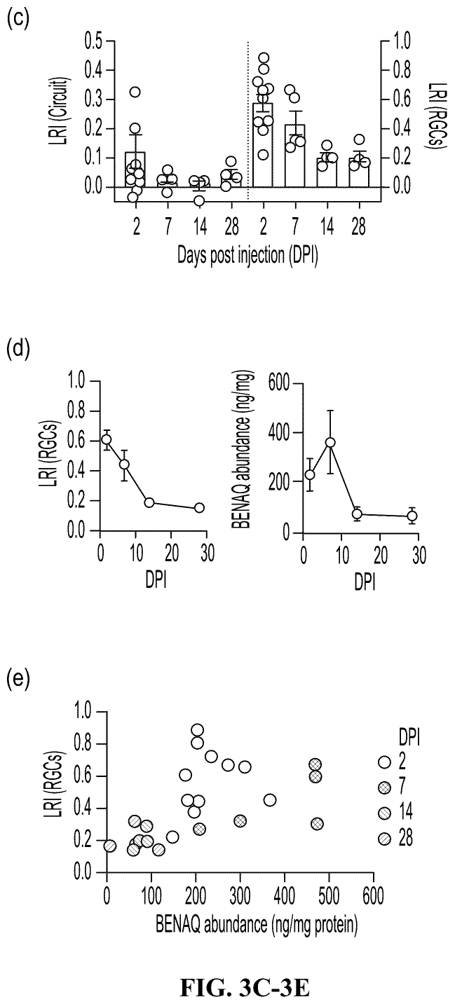

It was further discovered that a single in vivo injection of Compound 1 prepared in SBE-CD prolongs photosensitization of RGCs. FIG. 3a shows representative spike rasters and frequency plots from rd1 retina 2 and 28 days post-injection of 100 μM Compound 1 and 1200 μM SBE-CD. FIG. 3b shows spike rasters and frequency plots from the same tissue in FIG. 3a after pharmacological isolation of RGCs using a cocktail of synaptic blockers. FIG. 3c shows the time course of observed LRIs up to 28 days post-injection of Compound 1 and SBE-CD. FIG. 3d shows a comparison of the photosensitization time course with the time course of BENAQ uptake in rd1 retina. FIG. 3e is a scatter plot correlating the strength of light responses in RGCs and the abundance of Compound 1 in retinal tissue.

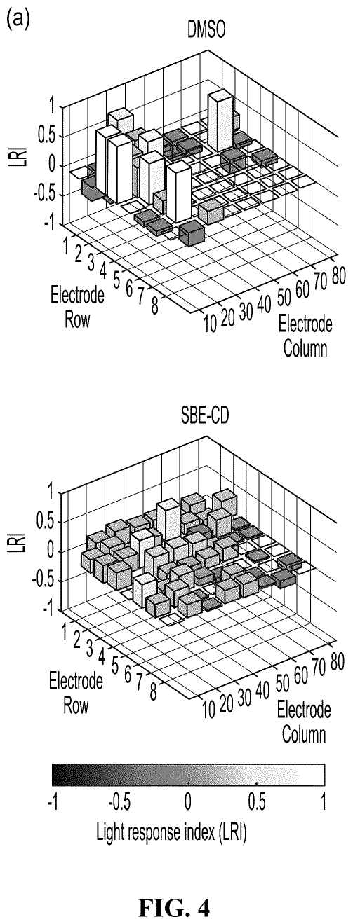

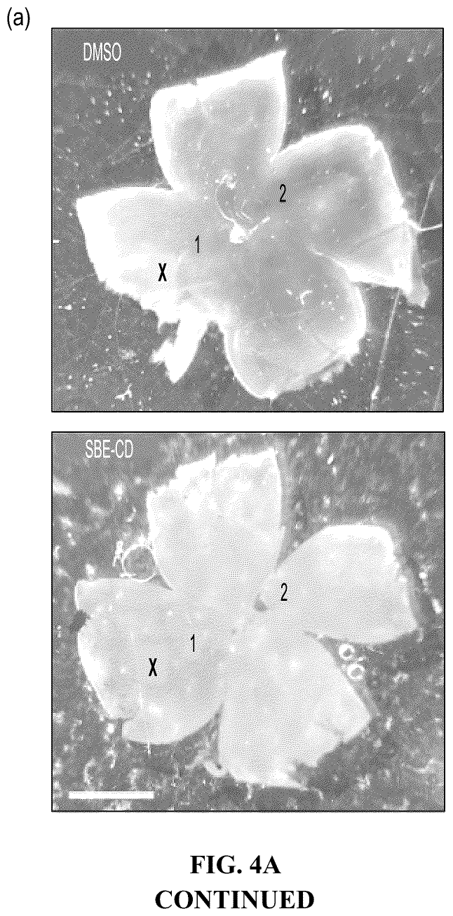

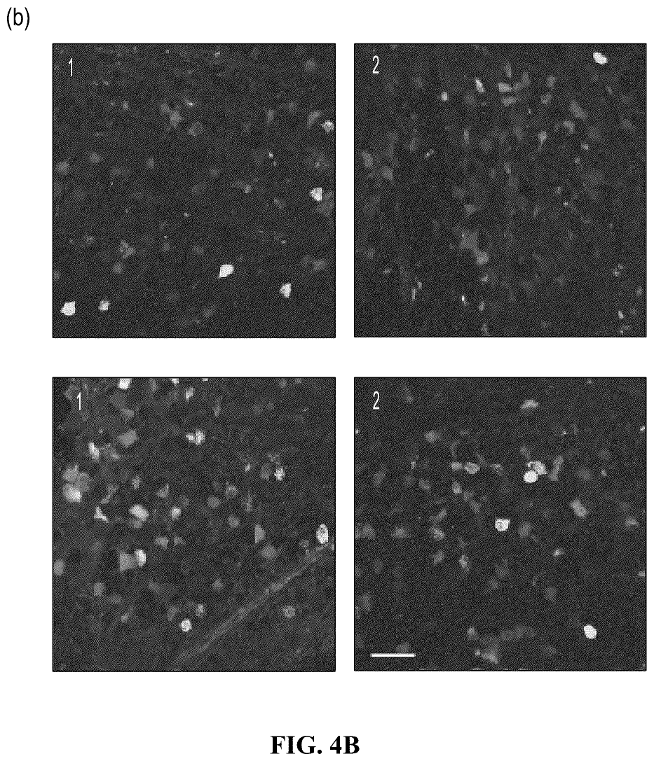

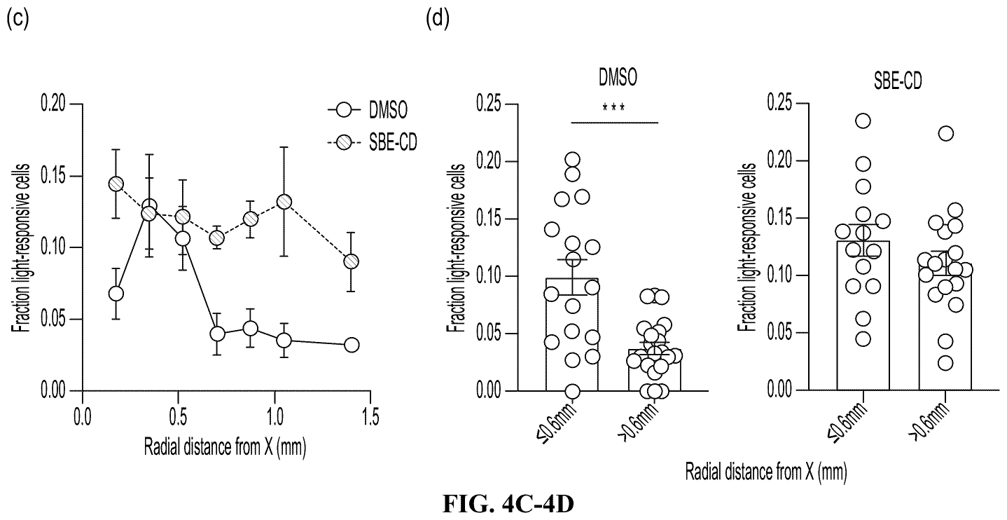

SBE-CD improved the dispersion of BENAQ to the retina. FIG. 4a shows a spatial LRI plots mapped to the MEA chip showing the distribution of light responses from Compound 1 injected with DMSO versus SBE-CD (left) and representative real-color pictures of rd1-GCaMP6f retina taken 2-days post injection with 100 μM Compound 1 in DMSO or 1200 μM SBE-CD (right) (X=location on the retina below the vitreal space where the injection bolus was applied. Scale bar=500 μm). FIG. 4b shows correlation micrographs obtained from the rd1-GCaMP6f retina shown in FIG. 4a. Two locations were imaged in both retinas corresponding to either 350 μm (region 1) or 1050 μm (region 2) away from X (scale bar=300 μm). FIG. 4c is a plot of the fraction of light-responsive cells against the radial distance from X in a DMSO-based or SBE-CD-based injection of Compound 1. FIG. 4d shows bar plots demonstrating the consistency of light-responses past 600 μm in DMSO vs in SBE-CD.

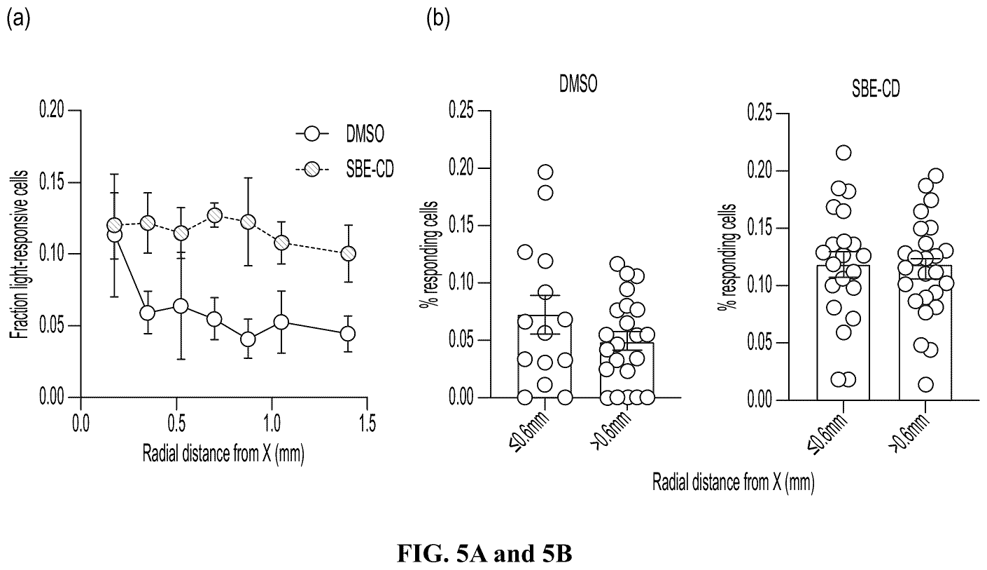

2-photon imaging 2 weeks post-injection shows persistent photosensitization using SBE-CD. FIG. 5a is a plot of light-responsive cells as a function of distance from injection site X 2 weeks after injection using DMSO or SBE-CD. FIG. 5b shows bar plots demonstrating the consistency of light-responses past 600 μm in DMSO vs in SBE-CD.

What is claimed is:

1. A pharmaceutical composition, comprising:
an alkylated cyclodextrin; and
a compound of formula (I): 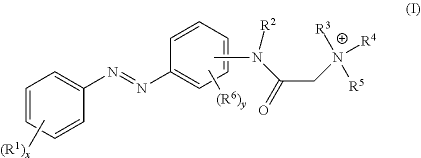
wherein each of R₁ are independently selected from C₁₋₁₀ alkyl, substituted C₁₋₁₀ alkyl, —NR¹⁰R¹¹, —NR¹²C(O)R¹³, C₂₋₁₀ alkenyl, substituted C₂₋₁₀ alkenyl, C₂ alkynyl, substituted C₂₋₁₀ alkynyl, C₆₋₂₀ aryl; substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, —C(O)OR¹⁰, and —S(O)₂R¹⁰;
x is an integer from 0 to 5;
y is an integer from 0 to 4;
R² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
R³, R⁴, and R⁵ are independently selected from hydrogen, C₂₋₈ alkyl, substituted C₂₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
each R⁶ is independently selected from hydrogen, C₁₋₁₀ alkyl, substituted C₁₋₁₀ alkyl, NR¹⁰R¹¹, —NR¹²C(O)R¹³, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, heteroaryl, heterocyclic, heterocyclooxy, heterocyclothio, heteroarylamino, heterocycloamino, C₄₋₁₀ cycloalkyl, substituted C₄₋₁₀ cycloalkyl, C₄₋₁₀ cycloalkenyl, substituted C₄₋₁₀ cycloalkenyl, cyano, halo, —OR¹⁰, —C(O)OR¹⁰, —S(O)R¹⁰, and —S(O)₂R¹⁰;
R¹⁰ and R¹¹ are independently selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl;
R¹² is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆₋₂₀ aryl, substituted C₆₋₂₀ aryl, C₄₋₁₀ cycloalkyl, substituted C₄₋₁₀ cycloalkyl, C₄₋₁₀ cycloalkenyl, and substituted C₄₋₁₀ cycloalkenyl; and
R¹³ is selected from hydrogen, C₁₋₁₀ alkyl, substituted C₁₋₁₀ alkyl, C₂₋₁₀ alkenyl, substituted C₂₋₁₀ alkenyl, C₂₋₁₀ alkynyl, substituted C₂₋₁₀ alkynyl, C₆-C₁₀ aryl, substituted C₆₋₂₀ aryl, C₄₋₁₀ cycloalkyl, substituted C₄₋₁₀ cycloalkyl, C₄₋₁₀ cycloalkenyl, substituted C₄₋₁₀ cycloalkenyl, —CH₂—N(CH₂CH₃)₃⁺, and —CH₂—SO₃⁻,
or a pharmaceutically acceptable salt thereof;
wherein the azo bond in the compound of formula (I) may be either cis or trans; and
wherein the molar ratio of cyclodextrin to compound of formula (I) is from 1:1 to 500:1.

2. The composition of claim 1, wherein the alkylated cyclodextrin has the structure of formula (II): 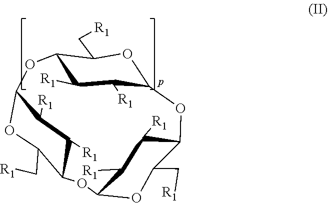
or pharmaceutically acceptable salts thereof, wherein p is 4, 5, or 6, and R₁ is independently selected at each occurrence from —OH and optionally substituted —O-C₁-C₈ alkyl, wherein at least one R₁ is an optionally substituted —O-C₁-C₈ alkyl.

3. The composition of any one of claims 1 to 2, wherein the average degree of substitution with optionally substituted —O-C₁-C₈ alkyl of all cyclodextrin molecules in the composition is between 4 and 9.

4. The composition of any one of claims 1 to 2, wherein the average degree of substitution with optionally substituted —O-C₁-C₈ alkyl of all cyclodextrin molecules in the composition is between 6.5 and 7.5.

5. The composition of any one of claims 2 to 4, wherein at least one R₁ is O-(C₂-C₈ alkylene)-SO₃⁻-T, wherein T is independently selected at each occurrence from pharmaceutically acceptable cations.

6. The composition of any one of claims 2 to 4, wherein at least one R₁ is —O-C₁-C₈ alkyl substituted with hydroxy.

7. The composition of any one of claims 2 to 6, wherein p is 5.

8. The composition of claim 1, wherein the cyclodextrin has the structure of formula (III). 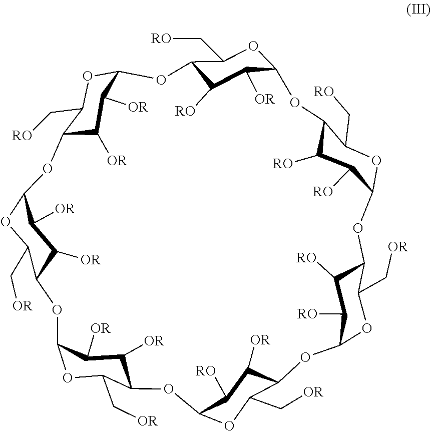
wherein each R is independently —H or —(CH₂)₄—SO₃⁻Na⁺, and the average degree of substitution with —(CH₂)₄—SO₃⁻Na⁺ of all cyclodextrin molecules in the composition is from 6 to 7.1.

9. The composition of claim 1, wherein the compound of formula (I) is selected from the group consisting of: 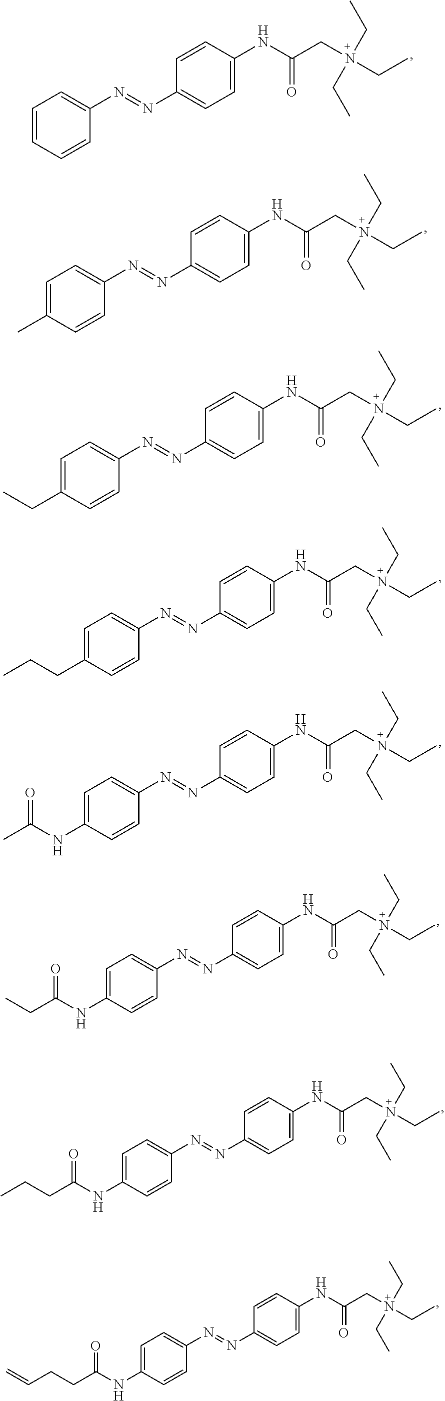 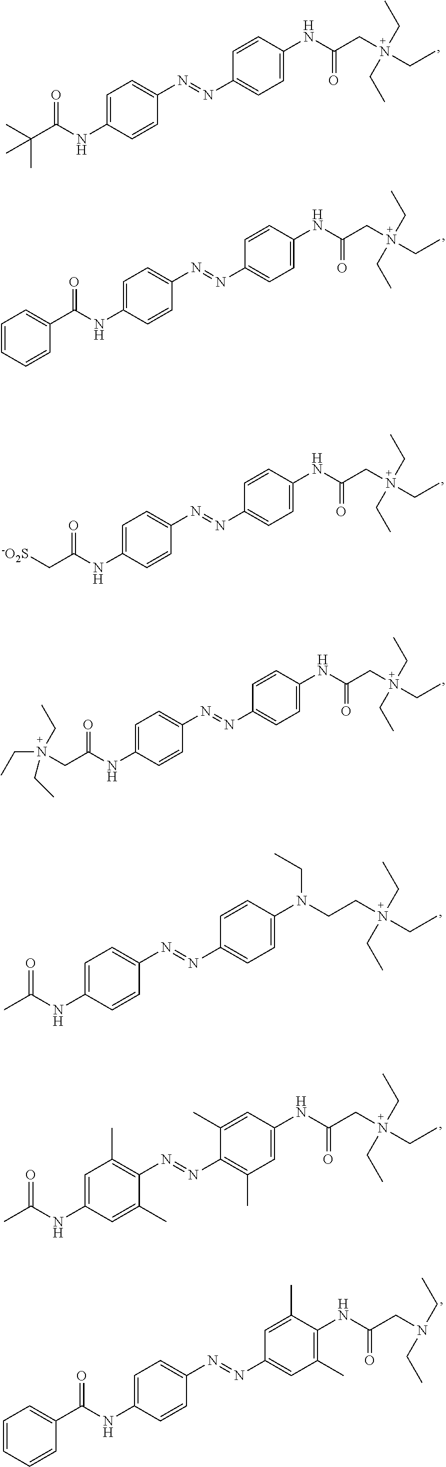 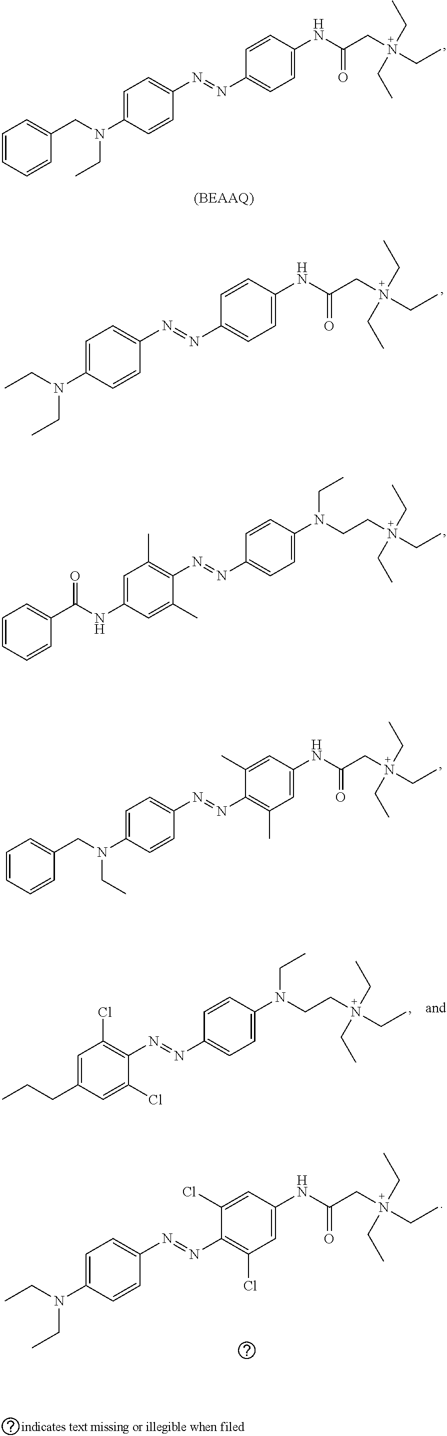
or pharmaceutically acceptable salts thereof, wherein the azo bond in the compounds of formula (I) may be either cis or trans.

10. The composition of any one of claims 1 to 8, wherein the compound of formula (I) has the structure: 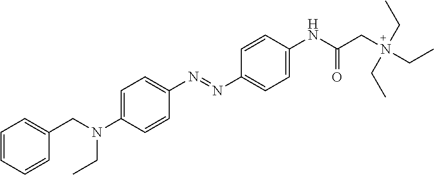
or a pharmaceutically acceptable salt thereof, wherein the azo bond in the structure may be either cis or trans.

11. The composition of any one of claims 1 to 10, wherein the molar ratio of cyclodextrin to compound of formula (I) is from 2:1 to 350:1.

12. The composition of any one of claims 1 to 10, wherein the molar ratio of cyclodextrin to compound of formula (I) is from 3:1 to 150:1.

13. The composition of any one of claims 1 to 12, wherein the concentration of the compound of formula (I) is from 50 μM to 200 μM.

14. The composition of any one of claims 1 to 12, wherein the concentration of the compound of formula (I) is from 75 μM to 150 μM.

15. The composition of any one of claims 1 to 12, wherein the concentration of the compound of formula (I) is about 100 μM.

16. The composition of any one of claims 1 to 15, wherein the azo bond in greater than 50% of molecules of formula (I) in the composition is trans.

17. The composition of any one of claims 1 to 15, wherein the azo bond in greater than 70% of molecules of formula (I) in the composition is trans.

18. The composition of any one of claims 1 to 15, wherein the azo bond in greater than 90% of molecules of formula (I) in the composition is trans.

19. The composition of any one of claims 1 to 15, wherein the azo bond in greater than 95% of molecules of formula (I) in the composition is trans.

20. The composition of any one of claims 1 to 15, wherein the azo bond in greater than 50% of molecules of formula (I) in the composition is cis.

21. The composition of any one of claims 1 to 15, wherein the azo bond in greater than 70% of molecules of formula (I) in the composition is cis.

22. The composition of any one of claims 1 to 15, wherein the azo bond in greater than 90% of molecules of formula (I) in the composition is cis.

23. The composition of any one of claims 1 to 15, wherein the azo bond in greater than 95% of molecules of formula (I) in the composition is cis.

24. The composition of any one of claims 1 to 15, wherein greater than 50% of the molecules of formula (I) in the composition are complexed with the cyclodextrin.

25. The composition of any one of claims 1 to 15, wherein greater than 70% of the molecules of formula (I) in the composition are complexed with the cyclodextrin.

26. The composition of any one of claims 1 to 15, wherein greater than 90% of the molecules of formula (I) in the composition are complexed with the cyclodextrin.

27. The composition of any one of claims 1 to 15, wherein greater than 95% of the molecules of formula (I) in the composition are complexed with the cyclodextrin.

28. The composition of any one of claims 1 to 15, wherein less than 50% of the molecules of formula (I) in the composition are complexed with the cyclodextrin.

29. The composition of any one of claims 1 to 15, wherein less than 30% of the molecules of formula (I) in the composition are complexed with the cyclodextrin.

30. The composition of any one of claims 1 to 15, wherein less than 10% of the molecules of formula (I) in the composition are complexed with the cyclodextrin.

31. The composition of any one of claims 1 to 30, further comprising a buffer.

32. The composition of any one of claims 1 to 31, wherein the composition is an aqueous solution.

33. The composition of any one of claims 1 to 31, wherein the solution has an osmolality between 250 mOsm and 350 mOsm.

34. The composition of any one of claims 1 to 31, wherein the composition is a solid suitable for reconstitution.

35. A method of treating a retinal disorder, comprising injecting the composition of any one of claims 1 to 33 into the vitreous of a subject having the retinal disorder.

36. The method of claim 35, wherein the retinal disorder is retinitis pigmenosa or age-related macular degeneration.

37. The method of any one of claims 35 to 36, comprising prior to said injection, exposing the composition to light.

38. The method of claim 37, wherein the light comprises light having a wavelength of 350 nm to 500 nm.

39. The method of any one of claims 37 to 38, wherein the composition is exposed to from 200 J/ml to 2000 J/ml of 350 nm to 500 nm light.
